(19) United States
(12) Patent Application Publication
(10) Pub. No.: US 2024/0049944 A1
(43) Pub. Date: Feb. 15, 2024

(54) Recording Medium, Method for Generating Learning Model, and Surgery Support Device

(71) Applicant: Anaut Inc., Tokyo (JP)

(72) Inventors: Nao Kobayashi, Tokyo (JP); Yuta Kumazu, Tokyo (JP); Seigo Senya, Tokyo (JP)

(21) Appl. No.: 18/268,889

(22) PCT Filed: Dec. 27, 2021
(86) PCT No.: PCT/JP2021/048592

§ 371 Date: Jun. 21, 2023

(30) Foreign Application Priority Data
Dec. 29, 2020 (JP) 2020-219806

(51) Int. Cl. A61B 1/00; A61B 1/06

(52) U.S. Cl. CPC A61B 1/0005; A61B 1/06; A61B 1/000096

(57) ABSTRACT

A computer program causes a computer to execute processing of acquiring an operation field image obtained by shooting an operation field of a scopic surgery, and distinctively recognizing blood vessels included in the acquired operation field image and a notable blood vessel among the blood vessels by using a learning model trained to output information relevant to a blood vessel when the operation field image is input.

### CROSS-REFERENCE TO RELATED APPLICATIONS

This application is the national phase under 35 U. S. C. § 371 of PCT International Application No. PCT/JP2021/048592 which has an International filing date of Dec. 27, 2021 and designated the United States of America.

### FIELD

The present invention relates to a recording medium, a method for generating a learning model, and a surgery support device.

### BACKGROUND

In a laparoscopic surgery, for example, a surgery for removing an affected area such as a malignant tumor that is formed in the body of a patient is performed.

In this case, the inside of the body of the patient is shot with a laparoscope, and the obtained operation field image is displayed on a monitor (for example, refer to Japanese Patent Laid-Open Publication No. 2005-287839).

In the related art, it was difficult to recognize a blood vessel that requires the attention of a surgeon from the operation field image, and to notify the recognition of the blood vessel to the surgeon.

### SUMMARY

An object of the present application is to provide a recording medium, a method for generating a learning model, and a surgery support device, in which it is possible to output a recognition result of a blood vessel from an operation field image.

A non-transitory computer readable recording medium in one aspect of the present application stores a computer program for causing a computer to execute processing of acquiring an operation field image obtained by shooting an operation field of a scopic surgery, and distinctively recognizing blood vessels included in the acquired operation field image and a notable blood vessel among the blood vessels by using a learning model trained to output information relevant to a blood vessel when the operation field image is input.

A method for generating a learning model in one aspect of the present application is a method for generating a learning model for causing a computer to execute processing of acquiring training data including an operation field image obtained by shooting an operation field of a scopic surgery, first ground truth data indicating blood vessel portions included in the operation field image, and second ground truth data indicating a notable blood vessel among the blood vessel portions, and generating a learning model for outputting information relevant to a blood vessel, on the basis of a set of the acquired training data, when the operation field image is input.

A surgery support device in one aspect of the present application includes a processor and a storage storing instructions causing the processor to execute processes of acquiring an operation field image obtained by shooting an operation field of a scopic surgery, distinctively recognizing blood vessels included in the acquired operation field image and a notable blood vessel among the blood vessels by using a learning model trained to output information relevant to a blood vessel when the operation field image is input, and outputting support information relevant to the scopic surgery, on the basis of a recognition result.

According to the present application, it is possible to output the recognition result of the blood vessel from the operation field image. The above and further objects and features of the invention will more fully be apparent from the following detailed description with accompanying drawings.

### BRIEF DESCRIPTION OF THE DRAWINGS

FIG. 1 is a schematic view describing a schematic configuration of a laparoscopic surgery support system according to Embodiment 1;

FIG. 2 is a block diagram describing an internal configuration of a surgery support device;

FIG. 3 is a schematic view illustrating an example of an operation field image;

FIG. 4 is a schematic view illustrating a configuration example of a first learning model;

FIG. 5 is a schematic view illustrating a recognition result of the first learning model;

FIG. 6 is a schematic view illustrating a configuration example of a second learning model;

FIG. 7 is a schematic view illustrating a recognition result of the second learning model;

FIG. 8 is a flowchart describing a generation procedure of the first learning model;

FIG. 9 is a flowchart describing an execution procedure of surgery support;

FIG. 10 is a schematic view illustrating a display example of a small blood vessel;

FIG. 11 is a schematic view illustrating a display example of a notable blood vessel;

FIG. 12 is an explanatory diagram describing a method for generating training data for the second learning model;

FIG. 13 is an explanatory diagram describing a configuration of a softmax layer of a learning model in Embodiment 3;

FIG. 14 is a schematic view illustrating a display example in Embodiment 3;

FIG. 15 is a schematic view illustrating a display example in Embodiment 4;

FIG. 16 is an explanatory diagram describing a display method in Embodiment 5;

FIG. 17 is a flowchart illustrating a procedure of processing that is executed by a surgery support device according to Embodiment 6;

FIG. 18 is a schematic view illustrating a display example in Embodiment 6;

FIG. 19 is an explanatory diagram describing a configuration of a softmax layer of a learning model in Embodiment 7;

FIG. 20 is a schematic view illustrating a display example in Embodiment 7;

FIG. 21 is a schematic view illustrating a configuration example of a learning model for a special light image;

FIG. 22 is a flowchart describing a procedure of processing that is executed by a surgery support device according to Embodiment 8;

FIG. 23 is an explanatory diagram describing an outline of processing that is executed by a surgery support device according to Embodiment 9;

FIG. 24 is a flowchart describing an execution procedure of surgery support in Embodiment 10;

FIG. 25 is a schematic view illustrating an example of enlarged display; and

FIG. 26 is a schematic view illustrating an example of warning display.

### DESCRIPTION OF EMBODIMENTS

Hereinafter, a form in which the present invention is applied to a support system of a laparoscopic surgery will be described in detail by using the drawings. Note that, the present invention is not limited to the laparoscopic surgery, and can be applied to the general scopic surgery using an imaging device such as a thoracoscope, an intestinal endoscope, a cystoscope, an arthroscope, a robot-supported endoscope, a surgical microscope, and an exoscope.

### Embodiment 1

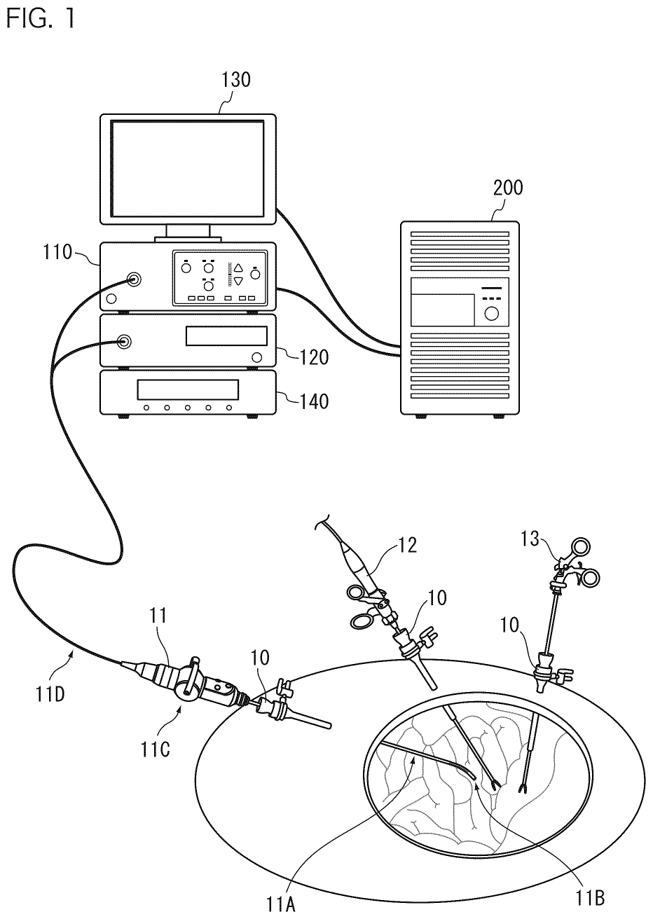

FIG. 1 is a schematic view illustrating a schematic configuration of a laparoscopic surgery support system according to Embodiment 1. In a laparoscopic surgery, instead of performing a laparotomy, a plurality of tools for a stoma referred to as a trocar 10 are attached to the abdominal wall of a patient, and tools such as a laparoscope 11, an energy treatment tool 12, and forceps 13 are inserted to the inside of the body of the patient from the stoma provided in the trocar 10. A surgeon performs a treatment such as the excision of an affected area by using the energy treatment tool 12 while looking at an image (an operation field image) of the inside of the body of the patient, which is shot by the laparoscope 11, in real time. The surgical tools such as the laparoscope 11, the energy treatment tool 12, and the forceps 13 are retained by the surgeon, a robot, or the like. The surgeon is a medical service worker associated with the laparoscopic surgery, and includes an operating surgeon, an assistant, a nurse, a medical doctor monitoring a surgery, and the like.

The laparoscope 11 includes an insertion portion 11A inserted to the inside of the body of the patient, an imaging device 11B built in the tip portion of the insertion portion 11A, a manipulation unit 11C provided in the end portion of the insertion portion 11A, and a universal code 11D for connecting to a camera control unit (CCU) 110 or a light source device 120.

The insertion portion 11A of the laparoscope 11 is formed of a rigid tube. A bent portion is provided in the tip portion of the rigid tube. A bending mechanism in the bent portion is a known mechanism built in the general laparoscope, and is configured to be bent, for example, in four directions of the left, right, top, and bottom by the tugging of a manipulation wire coupled to the manipulation of the manipulation unit 11C. Note that, the laparoscope 11 is not limited to a soft scope including the bent portion as described above, but may be a rigid scope not including the bent portion, or may be an imaging device not including the bent portion or the rigid tube. Further, the laparoscope 11 may be a 360-degree camera shooting a 360-degree range.

The imaging device 11B includes a driver circuit including a solid state image sensor such as a complementary metal oxide semiconductor (CMOS), a timing generator (TG), an analog signal processing circuit (AFE), and the like. The driver circuit of the imaging device 11B imports each of RGB signals output from the solid state image sensor in synchronization with a clock signal output from the TG, performs required processing such as noise removal, amplification, and AD conversion in the AFE, and generates image data in a digital format. The driver circuit of the imaging device 11B transmits the generated image data to the CCU 110 through the universal code 11D.

The manipulation unit 11C includes an angle lever, a remote switch, or the like, which is manipulated by the surgeon. The angle lever is a manipulation tool for receiving a manipulation for bending the bent portion. Instead of the angle lever, a bending manipulation knob, a joystick, and the like may be provided. The remote switch, for example, includes a switching switch for switching an observation image to moving image display or still image display, a zoom switch for zooming in or out the observation image, and the like. A specific function set in advance may be allocated to the remote switch, or a function set by the surgeon may be allocated to the remote switch.

In addition, a vibrator including a linear resonant actuator, a piezo actuator, or the like may be built in the manipulation unit 11C. In a case where there is an event to be notified to the surgeon manipulating the laparoscope 11, the CCU 110 may vibrate the manipulation unit 11C by operating the vibrator built in the manipulation unit 11C to notify the surgeon of the occurrence of the event.

In the insertion portion 11A, the manipulation unit 11C, and the universal code 11D of the laparoscope 11, a transmission cable for transmitting a control signal output to the imaging device 11B from the CCU 110 or image data output from the imaging device 11B, a light guide for guiding illumination light exiting from the light source device 120 to the tip portion of the insertion portion 11A, and the like are arranged. The illumination light exiting from the light source device 120 is guided to the tip portion of the insertion portion 11A through the light guide, and is applied to an operation field through an illumination lens provided in the tip portion of the insertion portion 11A. Note that, in this embodiment, the light source device 120 is described as an independent device, but the light source device 120 may be built in the CCU 110.

The CCU 110 includes a control circuit for controlling the operation of the imaging device 11B provided in the laparoscope 11, an image processing circuit for processing the image data from the imaging device 11B that is input through the universal code 11D, and the like. The control circuit includes a central processing unit (CPU), a read only memory (ROM), a random access memory (RAM), and the like, outputs the control signal to the imaging device 11B, in accordance with the manipulation of various switches provided in the CCU 110 or the manipulation of the manipulation unit 11C provided in the laparoscope 11, and performs control such as shooting start, shooting stop, and zooming. The image processing circuit includes a digital signal processor (DSP), an image memory, and the like, and performs suitable processing such as color separation, color interpolation, gain correction, white balance adjustment, and gamma correction, with respect to the image data input through the universal code 11D. The CCU 110 generates a frame image for a moving image from the image data after the processing, and sequentially outputs each of the generated frame images to a surgery support device 200 described below. The frame rate of the frame image, for example, is 30 frames per second (FPS).

The CCU 110 may generate video data based on a predetermined standard such as a national television system committee (NTSC), a phase alternating line (PAL), and digital imaging and communication in medicine (DICOM). The CCU 110 outputs the generated video data to a display device 130, and thus, is capable of displaying the operation field image (a video) on a display screen of the display device 130 in real time. The display device 130 is a monitor including a liquid crystal panel, an organic electro-luminescence (EL) panel, or the like. In addition, the CCU 110 may output the generated video data to a recording device 140 to record the video data in the recording device 140. The recording device 140 includes a recording device such as a hard disk drive (HDD) that records the video data output from the CCU 110, together with an identifier for identifying each surgery, surgery date and time, a surgery site, a patient name, a surgeon name, and the like.

The surgery support device 200 generates support information relevant to a laparoscopic surgery, on the basis of the image data input from the CCU 110 (that is, the image data of the operation field image obtained by shooting the operation field). Specifically, the surgery support device 200 performs processing of distinctively recognizing all small blood vessels included in the operation field image and a small blood vessel to be noticed among these small blood vessels to display information relevant to the recognized small blood vessel on the display device 130.

In this embodiment, an intrinsic name is not applied to the small blood vessel, and the small blood vessel represents a small blood vessel irregularly running the inside of the body. A blood vessel easily recognizable by the surgeon to which an intrinsic name is applied may be excluded from a recognition target. That is, the blood vessel to which the intrinsic name is applied, such as a left gastric artery, a right gastric artery, a left hepatic artery, a right hepatic artery, a splenic artery, a superior mesenteric artery, an inferior mesenteric artery, a hepatic vein, a left renal vein, and a right renal vein may be excluded from the recognition target. The small blood vessel is a blood vessel with a diameter of approximately 3 mm or less. A blood vessel with a diameter of greater than 3 mm can also be the recognition target insofar as an intrinsic name is not applied to the blood vessel. On the contrary, a blood vessel with a diameter of 3 mm or less may be excluded from the recognition target in a case where an intrinsic name is applied to the blood vessel and the blood vessel is easily recognizable by the surgeon.

On the other hand, the small blood vessel to be noticed represents a blood vessel that requires the surgeon to pay attention (hereinafter, also referred to as a notable blood vessel) among the small blood vessels described above. The notable blood vessel is a blood vessel that may be damaged during the surgery or a blood vessel that may be ignored by the surgeon during the surgery. The surgery support device 200 may recognize a small blood vessel existing in the central visual field of the surgeon as the notable blood vessel, or may recognize a small blood vessel not existing in the central visual field of the surgeon as the notable blood vessel. In addition, the surgery support device 200 may recognize a small blood vessel in a state of tension such as stretching, as the notable blood vessel, regardless of the existence in the central visual field.

In this embodiment, a configuration will be described in which the surgery support device 200 executes the recognition processing of the small blood vessel, but the same function as that of the surgery support device 200 may be provided in the CCU 110, and the CCU 110 may execute the recognition processing of the small blood vessel.

Hereinafter, the internal configuration of the surgery support device 200, and recognition processing and display processing that are executed by the surgery support device 200 will be described.

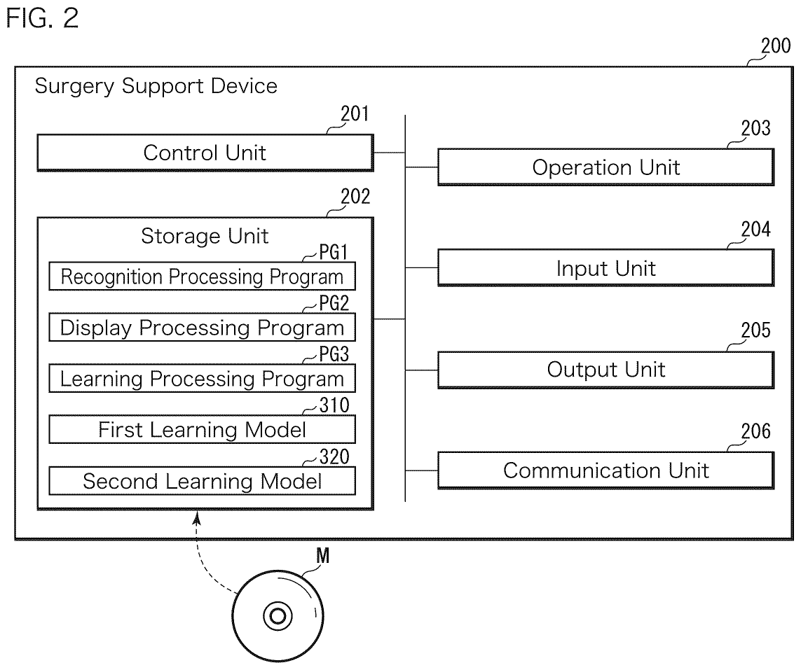

FIG. 2 is a block diagram illustrating the internal configuration of the surgery support device 200. The surgery support device 200 is a dedicated or general-purpose computer including a control unit 201, a storage unit 202, an operation unit 203, an input unit 204, an output unit 205, a communication unit 206, and the like. The surgery support device 200 may be a computer installed inside a surgery room, or may be a computer installed outside the surgery room. In addition, the surgery support device 200 may be a server installed inside a hospital in which the laparoscopic surgery is performed, or may be a server installed outside the hospital.

The control unit 201, for example, includes a CPU, a ROM, a RAM, and the like. In the ROM provided in the control unit 201, a control program and the like for controlling the operation of each hardware unit provided in the surgery support device 200 are stored. The CPU in the control unit 201 executes the control program stored in the ROM and various computer programs stored in the storage unit 202 described below, and controls the operation of each hardware unit, and thus, allows the entire device to function as the surgery support device in the present application. In the RAM provided in the control unit 201, data and the like used during the execution of operation are temporarily stored.

In this embodiment, a configuration has been described in which the control unit 201 includes the CPU, the ROM, and the RAM, but the configuration of the control unit 201 is optional, and for example, the control unit may be an arithmetic circuit or a control circuit including one or a plurality of graphics processing units (GPU), digital signal processors (DSP), field programmable gate arrays (FPGA), quantum processors, or volatile or non-volatile memories. In addition, the control unit 201 may have the function of a clock for outputting date and time information, a timer for measuring an elapsed time from the application of a measurement start instruction to the application of a measurement end instruction, a counter for counting numbers, or the like.

The storage unit 202 includes a storage device using a hard disk, a flash memory, or the like. In the storage unit 202, the computer program executed by the control unit 201, various data acquired from the outside, various data pieces generated in the device, and the like are stored.

The computer program stored in the storage unit 202 includes a recognition processing program PG1 for causing the control unit 201 to execute processing for recognizing a small blood vessel portion included in the operation field image, a display processing program PG2 for causing the control unit 201 to execute processing for displaying support information based on a recognition result on the display device 130, and a learning processing program PG3 for generating learning models 310 and 320. Note that, it is not necessary that the recognition processing program PG1 and the display processing program PG2 are computer programs independent from each other, and the programs may be implemented as one computer program. Such programs, for example, are provided by a non-transitory recording medium M in which the computer program is recorded to be readable. The recording medium M is a portable memory such as a CD-ROM, a USB memory, and a secure digital (SD) card. The control unit 201 reads a desired computer program from the recording medium M by using a reader that is not illustrated, and stores the read computer program in the storage unit 202. Alternatively, the computer program described above may be provided by communication using the communication unit 206.

In addition, in the storage unit 202, the learning models 310 and 320 used in the recognition processing program PG1 described above are stored. The learning model 310 is a learning model trained to output a recognition result of the small blood vessel portion included in the operation field image, with respect to the input of the operation field image. On the other hand, the learning model 320 is a learning model trained to output a recognition result of the small blood vessel portion to be noticed among the small blood vessels included in the surgeon image. Hereinafter, in the case of distinctively describing the learning models 310 and 320, the former will also be referred to as a first learning model 310, and the latter will also be referred to as a second learning model 320.

Each of the learning models 310 and 320 is described by definition information. The definition information of the learning models 310 and 320 includes information of layers in the learning models 310 and 320, information of nodes configuring each of the layers, and a parameter such as weighting and bias between the nodes. The learning model 310 stored in the storage unit 202 is a trained learning model that is trained by using a predetermined training algorithm with the operation field image obtained by shooting the operation field and ground truth data indicating the small blood vessel portion in the operation field image as training data. On the other hand, the learning model 320 is a trained learning model that is trained by using a predetermined training algorithm with the operation field image obtained by shooting the operation field and ground truth data indicating the notable blood vessel portion in the operation field image as training data. The configuration of the learning models 310 and 320 and a generation procedure of the learning models 310 and 320 will be described below in detail.

The operation unit 203 includes an operation device such as a keyboard, a mouse, a touch panel, and a stylus pen. The operation unit 203 receives the operation of the surgeon or the like, and outputs information relevant to the received operation to the control unit 201. The control unit 201 executes suitable processing, in accordance with operation information input from the operation unit 203. Note that, in this embodiment, a configuration has been described in which the surgery support device 200 includes the operation unit 203, but the operation may be received through various devices such as the CCU 110 connected to the outside.

The input unit 204 includes a connection interface for connecting an input device. In this embodiment, the input device connected to the input unit 204 is the CCU 110. The image data of the operation field image that is shot by the laparoscope 11 and is subjected to the processing by the CCU 110 is input to the input unit 204. The input unit 204 outputs the input image data to the control unit 201. In addition, the control unit 201 may not store the image data acquired from the input unit 204 in the storage unit 202.

In the embodiment, a configuration will be described in which the image data of the operation field image is acquired from the CCU 110 through the input unit 204, and the image data of the operation field image may be acquired directly from the laparoscope 11, or the image data of the operation field image may be acquired by an image processing device (not illustrated) that is detachably mounted on the laparoscope 11. In addition, the surgery support device 200 may acquire the image data of the operation field image recorded in the recording device 140.

The output unit 205 includes a connection interface for connecting an output device. In this embodiment, the output device connected to the output unit 205 is the display device 130. In a case where information to be notified to the surgeon or the like, such as a recognition result of the learning models 310 and 320, is generated, the control unit 201 outputs the generated information to the display device 130 from the output unit 205 to display the information on the display device 130. In this embodiment, a configuration has been described in which the display device 130 is connected to the output unit 205 as the output device, but an output device such as a speaker outputting a sound may be connected to the output unit 205.

The communication unit 206 includes a communication interface for transmitting and receiving various data. The communication interface provided in the communication unit 206 is a communication interface based on a wired or wireless communication standard that is used in Ethernet (registered trademark) or WiFi (registered trademark). In a case where data to be transmitted is input from the control unit 201, the communication unit 206 transmits the data to be transmitted to a designated destination. In addition, in a case where data transmitted from an external device is received, the communication unit 206 outputs the received data to the control unit 201.

It is not necessary that the surgery support device 200 is a single computer, and the surgery support device 200 may be a plurality of computers or a computer system including peripheral devices. Further, the surgery support device 200 may be a virtual machine that is virtually constructed by software.

Next, the operation field image that is input to the surgery support device 200 will be described.

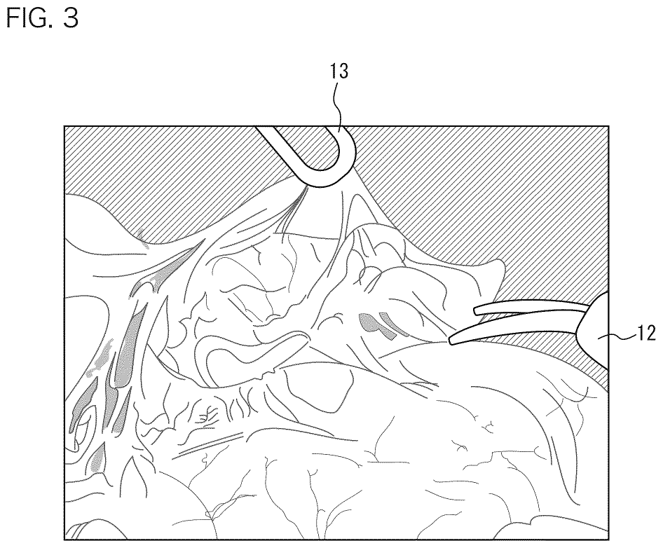

FIG. 3 is a schematic view illustrating an example of the operation field image. The operation field image in this embodiment is an image obtained by shooting the inside of the abdominal cavity of the patient with the laparoscope 11. It is not necessary that the operation field image is a raw image output from the imaging device 11B of the laparoscope 11, may be an image subjected to the processing by the CCU 110 or the like (the frame image).

The operation field shot with the laparoscope 11 includes tissues configuring internal organs, tissues including an affected area such as a tumor, a membrane or a layer covering the tissues, blood vessels existing around the tissues, and the like. The surgeon peels off or cuts off a target tissue by using a tool such as forceps or an energy treatment tool while grasping an anatomic structural relationship. The operation field image illustrated as an example in FIG. 3 illustrates a situation in which a membrane covering the internal organs is tugged by using the forceps 13, and the periphery of the target tissue including the membrane is peeled off by using the energy treatment tool 12. In a case where the blood vessel is damaged while the tugging or the peeling is performed, bleeding occurs. Tissue boundaries are blurred due to the bleeding, and it is difficult to recognize a correct peeling layer. In particular, the visual field is significantly degraded in a situation where hemostasis is difficult, and an excessive hemostasis manipulation causes a risk for a secondary damage.

In order to avoid the damage of the blood vessel, it is important to grasp a blood vessel structure, but the small blood vessel as described above is small and runs irregularly in general, and thus, it is not easy for the surgeon to grasp the blood vessel structure of the small blood vessel. Therefore, the surgery support device 200 according to this embodiment recognizes the small blood vessel portion included in the operation field image by using the learning models 310 and 320, and outputs the support information relevant to a laparoscopic surgery on the basis of the recognition result.

Next, a configuration example of the first learning model 310 and the second learning model 320 that are used in the surgery support device 200 will be described.

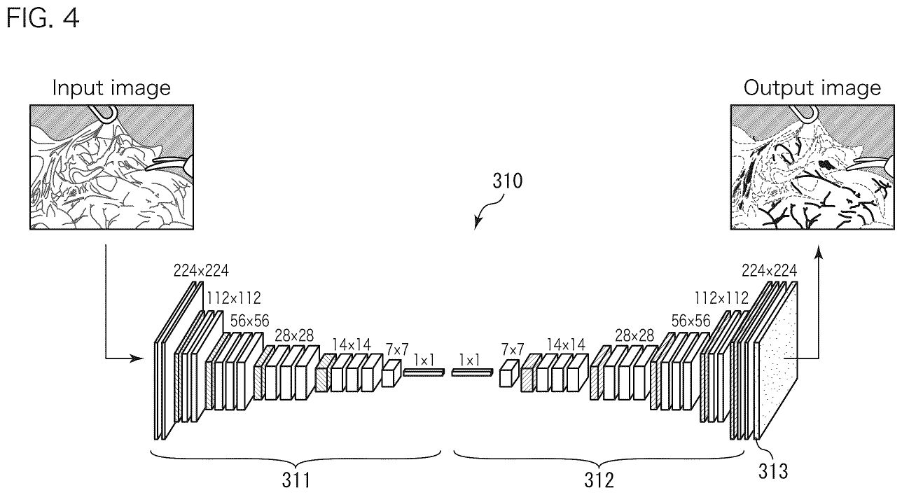

FIG. 4 is a schematic view illustrating a configuration example of the first learning model 310. The first learning model 310 is a learning model for performing image segmentation, and for example, is constructed by a neural network including a convolution layer such as SegNet. The first learning model 310 is not limited to SegNet, and may be configured by using any neural network such as a fully convolutional network (FCN), a U-shaped network (U-Net), and a pyramid scene parsing network (PSPNet), in which the image segmentation can be performed. In addition, the first learning model 310 may be constructed by using a neural network for object detection, such as you only look once (YOLO) and a single shot multi-box detector (SSD), instead of the neural network for image segmentation.

In this embodiment, an input image to the first learning model 310 is the operation field image obtained from the laparoscope 11. The first learning model 310 is trained to output an image indicating the recognition result of the small blood vessel portion included in the operation field image with respect to the input of the operation field image.

The first learning model 310, for example, includes an encoder 311, a decoder 312, and a softmax layer 313. The encoder 311 is configured such that a convolution layer and a pooling layer are alternately arranged. The convolution layer is multi-layered into two to three layers. In the example of FIG. 4, the convolution layer is illustrated without hatching, and the pooling layer is illustrated with hatching.

In the convolution layer, a convolution arithmetic operation between data to be input and a filter with a predetermined size (for example, 3×3, 5×5, or the like) is performed. That is, an input value input to a position corresponding to each element of the filter and a weight coefficient set in advance in the filter are multiplied for each element, and a linear sum of multiplication values for each element is calculated. By adding a set bias to the calculated linear sum, the output of the convolution layer is obtained. Note that, a result of the convolution arithmetic operation may be converted by an activating function. As the activating function, for example, a rectified linear unit (ReLU) can be used. The output of the convolution layer represents a feature map in which the feature of the input data is extracted.

In the pooling layer, a local statistic amount of the feature map output from the convolution layer that is a higher layer connected to the input side is calculated. Specifically, a window with a predetermined size corresponding to the position of the higher layer (for example, 2×2 or 3×3) is set, and the local statistic amount is calculated from the input value in the window. As the statistic amount, for example, the maximum value can be adopted. The size of the feature map output from the pooling layer is decreased (downsampled) in accordance with the size of the window. The example of FIG. 4 illustrates that the arithmetic operation in the convolution layer and the arithmetic operation in the pooling layer in the encoder 311 are sequentially repeated, and thus, an input image of 224 pixels×224 pixels is sequentially downsampled to feature maps of 112×112, 56×56, 28×28, . . . , and 1×1.

The output of the encoder 311 (in the example of FIG. 4, the feature map of 1×1) is input to the decoder 312. The decoder 312 is configured such that a deconvolution layer and an unpooling layer are alternately arranged. The deconvolution layer is multi-layered into two to three layers. In the example of FIG. 4, the deconvolution layer is illustrated without hatching, and the unpooling layer is illustrated with hatching.

In the deconvolution layer, a deconvolution arithmetic operation is performed with respect to the input feature map. The deconvolution arithmetic operation is an arithmetic operation for restoring the feature map before the convolution arithmetic operation under estimation that the input feature map is a result of performing the convolution arithmetic operation using a specific filter. In such an arithmetic operation, when the specific filter is represented by a matrix, a product between a transposed matrix with respect to the matrix and the input feature map is calculated, and thus, a feature map for output is generated. Note that, an arithmetic result of the deconvolution layer may be converted by the activating function such as ReLU as described above.

The unpooling layer provided in the decoder 312 is individually associated with the pooling layer provided in the encoder 311 on a one-to-one basis, and the associated pair has substantially the same size. In the unpooling layer, the size of the feature map downsampled in the pooling layer of the encoder 311 is increased (upsampled) again. The example of FIG. 4 illustrates that the arithmetic operation in the convolution layer and the arithmetic operation in the pooling layer in the decoder 312 are sequentially repeated, and thus, sequential upsampling is performed to feature maps of 1×1, 7×7, 14×14, . . . , and 224×224.

The output of the decoder 312 (in the example of FIG. 4, the feature map of 224×224) is input to the softmax layer 313. The softmax layer 313 applies a softmax function to an input value from the deconvolution layer connected to the input side, and thus, outputs the probability of a label for identifying a site in each position (pixel). In this embodiment, a label for identifying the small blood vessel may be set, and whether to belong to the small blood vessel may be identified by pixel unit. By extracting a pixel in which the probability of the label output from the softmax layer 313 is a threshold value or greater (for example, 70% or greater), an image indicating the recognition result of the small blood vessel portion (hereinafter, referred to as a recognition image) can be obtained.

Note that, in the example of FIG. 4, an image of 224 pixels×224 pixels is set as the input image to the first learning model 310, but the size of the input image is not limited to the above description, and can be suitably set in accordance with processing capability of the surgery support device 200, the size of the operation field image obtained from the laparoscope 11, and the like. In addition, it is not necessary that the input image to the first learning model 310 is the entire operation field image obtained from the laparoscope 11, and the input image may be a partial image generated by cutting out an attention area of the operation field image. The attention area including a treatment target is generally positioned in the vicinity of the center of the operation field image, and thus, for example, a partial image obtained by cutting out the vicinity of the center of the operation field image into the shape of a rectangle to have half the original size may be used. By decreasing the size of the image input to the first learning model 310, it is possible to increase a recognition accuracy while increasing a processing speed.

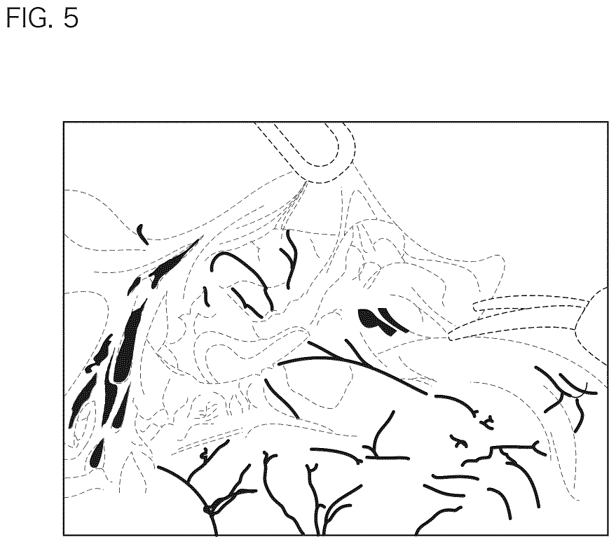

FIG. 5 is a schematic view illustrating the recognition result of the first learning model 310. In the example of FIG. 5, the small blood vessel portion recognized by using the first learning model 310 is illustrated with a thick solid line (or as an area painted with black), and other internal organs or membranes, and the portion of the surgical tool are illustrated with a broken line as a reference. The control unit 201 of the surgery support device 200 generates the recognition image of the small blood vessel for displaying the recognized small blood vessel portion to be discriminable. The recognition image is an image having the same size as that of the operation field image, in which a specific color is allocated to a pixel recognized as the small blood vessel. The color allocated to the small blood vessel is set arbitrarily. In addition, information indicating a transmittance is added to each pixel configuring the recognition image, a non-transmittance value is set to the pixel recognized as the small blood vessel, and a transmittance value is set to other pixels. The surgery support device 200 displays the recognition image generated as described above to be superimposed on the operation field image, and thus, is capable of displaying the small blood vessel portion on the operation field image as a structure with a specific color.

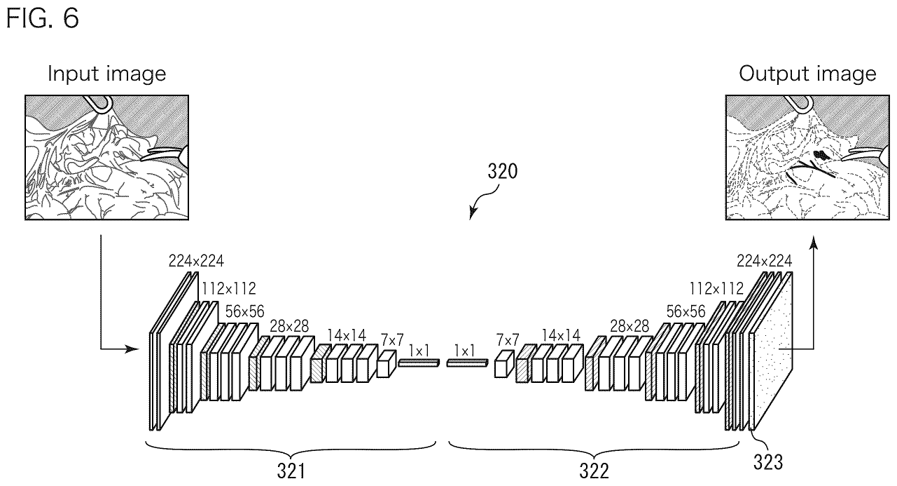

FIG. 6 is a schematic view illustrating a configuration example of the second learning model 320. The second learning model 320 includes an encoder 321, a decoder 322, and a softmax layer 323, and is configured to output an image indicating the recognition result of the notable blood vessel portion included in the operation field image with respect to the input of the operation field image. The configuration of the encoder 321, the decoder 322, and the softmax layer 323 that are provided in the second learning model 320 is the same as that of the first learning model 310, and thus, the detailed description thereof will be omitted.

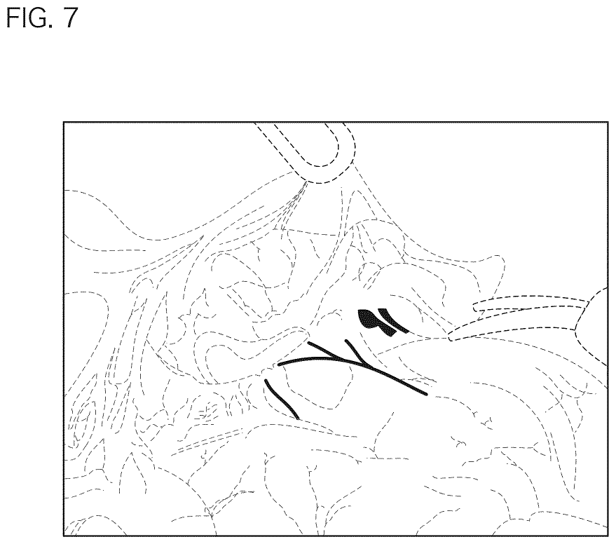

FIG. 7 is a schematic view illustrating the recognition result of the second learning model 320. In the example of FIG. 7, the notable blood vessel portion, which is recognized by using the second learning model 320, is illustrated with a thick solid line (or as an area painted with black), and the other internal organs or membranes, and the portion of the surgical tool are illustrated with a broken line as a reference. The control unit 201 of the surgery support device 200 generates the recognition image of the notable blood vessel for displaying the recognized notable blood vessel portion to be discriminable. The recognition image is an image having the same size as that of the operation field image, in which a specific color is allocated to a pixel recognized as the notable blood vessel. The color allocated to the notable blood vessel is different from the color allocated to the small blood vessel, and it is preferable that the color is distinguishable from the peripheral tissues. For example, the color allocated to the notable blood vessel may be a cool (blue-based) color such as blue or aqua, or may be a green-based color such as green or olive. In addition, information indicating a transmittance is added to each pixel configuring the recognition image, a non-transmittance value is set to the pixel recognized as the notable blood vessel, and a transmittance value is set to other pixels. The surgery support device 200 displays the recognition image generated as described above to be superimposed on the operation field image, and thus, is capable of displaying the notable blood vessel portion on the operation field image as a structure with a specific color.

Hereinafter, the generation procedure of the first learning model 310 and the second learning model 320 will be described. Annotation is performed with respect to the shot operation field image, as a preliminary stage for generating the first learning model 310 and the second learning model 320.

In the preliminary stage for generating the first learning model 310, an operator (an expert such as a medical doctor) performs the annotation by displaying the operation field image recorded in the recording device 140 on the display device 130 and designating a portion corresponding to the small blood vessel in pixel unit using the mouse, the stylus pen, or the like, which is provided as the operation unit 203. A set of a plurality of operation field images used in the annotation and data indicating the position of a pixel corresponding to the small blood vessel designated in each of the operation field images (first ground truth data) is stored in the storage unit 202 of the surgery support device 200 as training data for generating the first learning model 310. In order to increase the number of training data, a set of the operation field image generated by applying perspective conversion, reflective processing, or the like and ground truth data with respect to the operation field image may be included in the training data. Further, as the learning progresses, a set of the operation field image and the recognition result of the first learning model 310 obtained by inputting the operation field image (the ground truth data) may be included in the training data.

Similarly, in a preliminary stage for generating the second learning model 320, the operator performs the annotation by designating the small blood vessel existing in the central visual field of the surgeon (or the small blood vessel not existing in the central visual field of the surgeon) or a portion corresponding to the small blood vessel in a state of tension in pixel unit. The central visual field, for example, is a rectangular or circular area set in the center of the operation field image, and is set to have a size of approximately ¼ to ⅓ of the operation field image. A set of a plurality of operation field images used in the annotation and data indicating the position of a pixel corresponding to the notable blood vessel (second ground truth data), which is designated in each of the operation field images, is stored in the storage unit 202 of the surgery support device 200 as training data for generating the second learning model 320. In order to increase the number of training data, a set of the operation field image generated by applying perspective conversion, reflective processing, or the like and ground truth data with respect to the operation field image may be included in the training data. Further, as the learning progresses, a set of the operation field image and the recognition result of the second learning model 320 obtained by inputting the operation field image (the ground truth data) may be included in the training data.

The surgery support device 200 generates the first learning model 310 and the second learning model 320 by using the training data as described above.

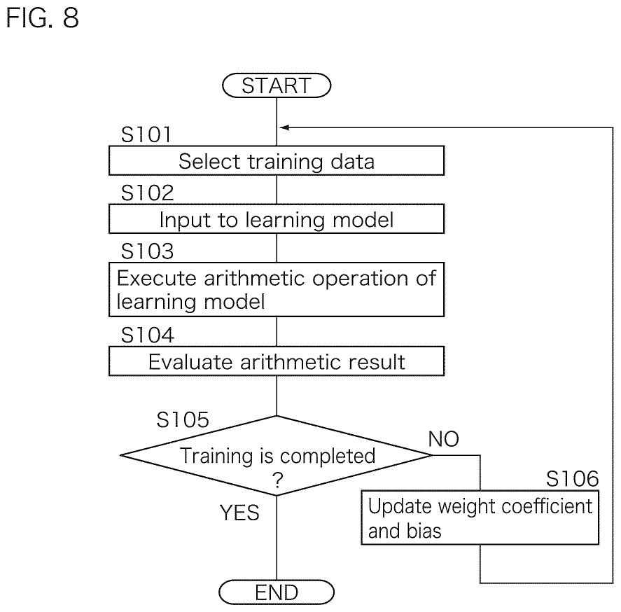

FIG. 8 is a flowchart illustrating the generation procedure of the first learning model 310. The control unit 201 of the surgery support device 200 reads out the learning processing program PG3 from the storage unit 202, and executes the following procedure, and thus, generates the first learning model 310. Note that, in a stage before the training is started, the initial value is applied to the definition information for describing the first learning model 310.

The control unit 201 accesses the storage unit 202, and selects a set of training data from the training data prepared in advance in order to generate the first learning model 310 (step S101). The control unit 201 inputs the operation field image included in the selected training data to the first learning model 310 (step S102), and executes an arithmetic operation of the first learning model 310 (step S103). That is, the control unit 201 generates the feature map from the input operation field image, and executes an arithmetic operation of the encoder 311 for sequentially downsampling the generated feature map, an arithmetic operation of the decoder 312 for sequentially upsampling the feature map input from the encoder 311, and an arithmetic operation of the softmax layer 313 for identifying each pixel of the feature map finally obtained by the decoder 312.

The control unit 201 acquires an arithmetic result from the first learning model 310, and evaluates the acquired arithmetic result (step S104). For example, the control unit 201 may calculate the degree of similarity between the image data of the small blood vessel obtained as the arithmetic result and the ground truth data included in the training data to evaluate the arithmetic result. The degree of similarity, for example, is calculated by a Jaccard coefficient. When the small blood vessel portion extracted by the first learning model 310 is set to A, and the small blood vessel portion included in the ground truth data is set to B, the Jaccard coefficient is applied by A∩B/A∪B×100(%). Instead of the Jaccard coefficient, a Dice coefficient or a Simpson coefficient may be calculated, or the degree of similarity may be calculated by using other existing methods.

The control unit 201 determines whether the training is completed, on the basis of the evaluation of the arithmetic result (step S105). In a case where the degree of similarity is greater than or equal to a threshold value set in advance, the control unit 201 is capable of determining that the training is completed.

In a case where it is determined that the training is not completed (S105: NO), the control unit 201 sequentially updates a weight coefficient and a bias in each layer of the first learning model 310 toward the input side from the output side of the learning model 310 by using an error back propagation algorithm (step S106). The control unit 201 updates the weight coefficient and the bias in each layer, and then, returns the processing to step S101, and executes again the processing of step S101 to step S105.

In a case where it is determined that the training is completed in step S105 (S105: YES), the trained first learning model 310 is obtained, and thus, the control unit 201 ends the processing of this flowchart.

In the flowchart of FIG. 8, the generation procedure of the first learning model 310 has been described, and the same applies to the generation procedure of the second learning model 320. That is, the surgery support device 200 may generate the second learning model 320 by repeatedly executing an arithmetic operation of the second learning model 320 and the evaluation of an arithmetic result using the training data prepared in order to generate the second learning model 320.

In this embodiment, the learning models 310 and 320 are generated in the surgery support device 200, but the learning models 310 and 320 may be generated by using an external computer such as a server device. The surgery support device 200 may acquire the learning models 310 and 320 generated in the external computer by using means such as communication, and may store the acquired learning models 310 and 320 in the storage unit 202.

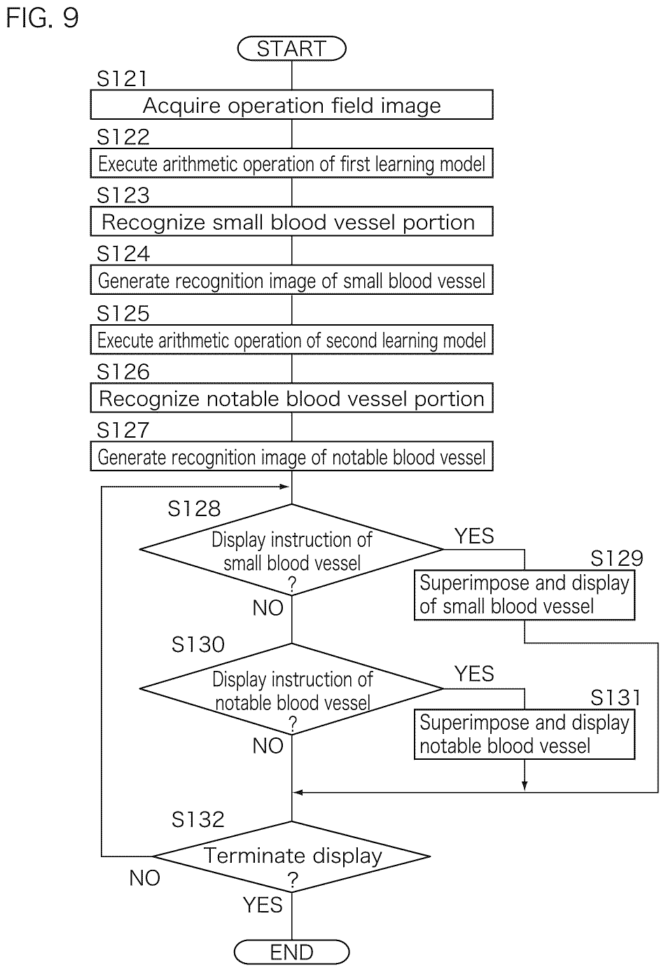

The surgery support device 200 performs surgery support in an operation phase after the learning models 310 and 320 are generated. FIG. 9 is a flowchart illustrating an execution procedure of the surgery support. The control unit 201 of the surgery support device 200 reads out the recognition processing program PG1 and the display processing program PG2 from the storage unit 202, and executes the programs, and thus, executes the following procedure. In a case where the laparoscopic surgery is started, the operation field image obtained by shooting the operation field with the imaging device 11B of the laparoscope 11 is output to the CCU 110 through the universal code 11D, as needed. The control unit 201 of the surgery support device 200 acquires the operation field image output from the CCU 110 in the input unit 204 (step S121). The control unit 201 executes the processing of step S122 to S127 each time when the operation field image is acquired.

The control unit 201 inputs the acquired operation field image to the first learning model 310 to execute the arithmetic operation of the first learning model 310 (step S122), and recognizes the small blood vessel portion included in the operation field image (step S123). That is, the control unit 201 generates the feature map from the input operation field image, and executes the arithmetic operation of the encoder 311 for sequentially downsampling the generated feature map, the arithmetic operation of the decoder 312 for sequentially upsampling the feature map input from the encoder 311, and the arithmetic operation of the softmax layer 313 for identifying each pixel of the feature map finally obtained by the decoder 312. In addition, the control unit 201 recognizes the pixel output from the softmax layer 313, in which the probability of the label is the threshold value or greater (for example, 70% or greater), as the small blood vessel portion.

In order to display the small blood vessel portion recognized by using the first learning model 310 to be discriminable, the control unit 201 generates the recognition image of the small blood vessel (step S124). The control unit 201, as described above, may allocate a specific color to the pixel recognized as the small blood vessel, and may set a transmittance to the pixels other than the small blood vessel such that the background is transmissive.

Similarly, the control unit 201 inputs the acquired operation field image to the second learning model 320 to execute the arithmetic operation of the second learning model 320 (step S125), and recognizes the notable blood vessel portion included in the operation field image (step S126). In a case where the annotation is performed such that the small blood vessel in the central visual field of the surgeon is recognized when the second learning model 320 is generated, in step S126, the small blood vessel existing in the central visual field of the surgeon is recognized as the notable blood vessel. In addition, in a case where the annotation is performed such that the small blood vessel not in the central visual field of the surgeon is recognized, in step S126, the small blood vessel not in the central visual field of the surgeon is recognized as the notable blood vessel. Further, in a case where the annotation is performed such that the small blood vessel in a state of tension is recognized, in step S126, the small blood vessel is recognized as the notable blood vessel in a stage where the small blood vessel is in a state of tension from not in a state of tension.

Next, in order to display the notable blood vessel portion, which is recognized by using the second learning model 320, to be discriminable, the control unit 201 generates the recognition image of the notable blood vessel (step S127). The control unit 201, as described above, may allocate a color different from that of the other small blood vessel portions, such as a blue-based color or a green-based color, to the pixel recognized as the notable blood vessel, and may set a transmittance to the pixels other than the notable blood vessel such that the background is transmissive.

Next, the control unit 201 determines whether a display instruction of the small blood vessel is applied (step S128). The control unit 201 may determine whether the instruction of the surgeon is received through the operation unit 203 to determine whether the display instruction is applied. In a case where the display instruction of the small blood vessel is applied (S128: YES), the control unit 201 outputs the recognition image of the small blood vessel generated at this time to the display device 130 from the output unit 205, and displays the recognition image of the small blood vessel on the display device 130 to be superimposed on the operation field image (step S129). Note that, in the immediately preceding frame, in a case where the recognition image of the notable blood vessel is displayed to be superimposed, instead of the recognition image of the notable blood vessel, the recognition image of the small blood vessel may be displayed to be superimposed. Accordingly, the small blood vessel portion recognized by using the learning model 310 is displayed on the operation field image as a structure indicated with a specific color.

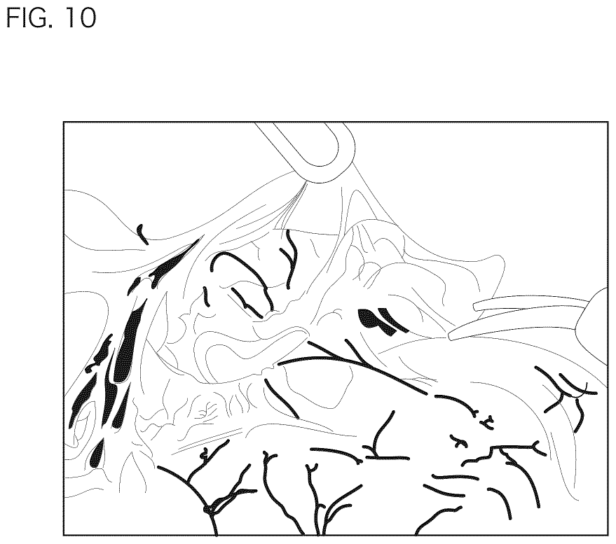

FIG. 10 is a schematic view illustrating a display example of the small blood vessel. For the convenience of drawing, in the display example of FIG. 10, the small blood vessel portion is illustrated with a thick solid line or as an area painted with black. In practice, since a portion corresponding to the small blood vessel is painted with a color set in advance in pixel unit, the surgeon is capable of recognizing the small blood vessel portion by checking the display screen of the display device 130.

In a case where it is determined that the display instruction of the small blood vessel is not applied (S128: NO), the control unit 201 determines whether a display instruction of the notable blood vessel is applied (step S130). The control unit 201 may determine whether the instruction of the surgeon is received through the operation unit 203 to determine whether the display instruction is applied. In a case where the display instruction of the notable blood vessel is applied (S130: YES), the control unit 201 outputs the recognition image of the notable blood vessel, which is generated at this point, to the display device 130 from the output unit 205, and displays the recognition image of the notable blood vessel to be superimposed on the operation field image on the display device 130 (step S131). Note that, in the immediately preceding frame, in a case where the recognition image of the small blood vessel is displayed to be superimposed, instead of the recognition image of the small blood vessel, the recognition image of the notable blood vessel may be displayed to be superimposed. Accordingly, the notable blood vessel, which is recognized by using the learning model 320, is displayed on the operation field image as a structure with a specific color such as a blue-based color or a green-based color.

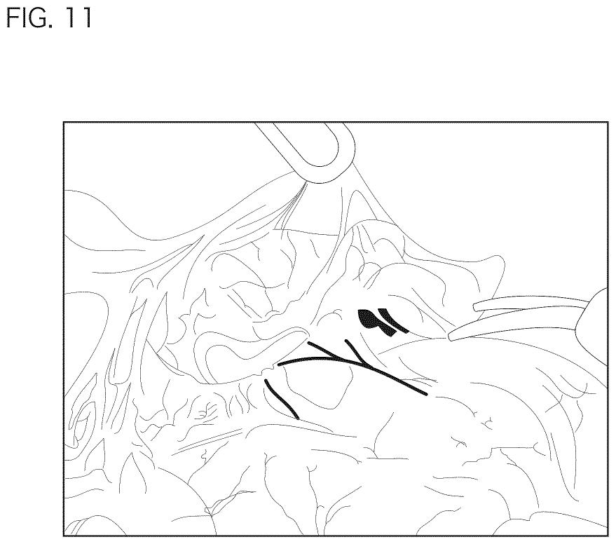

FIG. 11 is a schematic view illustrating a display example of the notable blood vessel. For the convenience of drawing, in the display example of FIG. 11, the notable blood vessel portion is illustrated with a thick solid line or as an area painted with black. In practice, since a portion corresponding to the notable blood vessel is painted with a color not existing inside the human body, such as a blue-based color or a green-based color, in pixel unit, the surgeon is capable of articulately determining the notable blood vessel by looking at the display screen of the display device 130. In a case where it is necessary to cut a site including the notable blood vessel, for example, the surgeon is capable of suppressing the occurrence of bleeding by performing clotting cutting with the energy treatment tool 12.

In a case where the display instruction of the notable blood vessel is not applied in step S130 (S130: NO), the control unit 201 determines whether to terminate the display of the operation field image (step S132). In a case where the laparoscopic surgery is ended, and the shooting of the imaging device 11B of the laparoscope 11 is stopped, the control unit 201 determines to terminate the display of the operation field image. In a case where it is determined not to terminate the display of the operation field image (S132: NO), the control unit 201 returns the processing to step S128. In a case where it is determined to terminate the display of the operation field image (S132: YES), the control unit 201 ends the processing of this flowchart.

In the flowchart illustrated in FIG. 9, the processing of recognizing the small blood vessel is executed, and then, the processing of recognizing the notable blood vessel is executed, but the execution orders thereof may be reversed, or may be simultaneously executed in parallel.

In addition, in the flowchart illustrated in FIG. 9, in a case where the display instruction of the small blood vessel is not applied, the recognition image of the small blood vessel may be displayed to be superimposed, and in a case where the display instruction of the notable blood vessel is applied, the recognition image of the notable blood vessel is displayed to be superimposed, but either the recognition image of the small blood vessel or the recognition image of the notable blood vessel may be displayed by default without receiving the display instruction. In this case, the control unit 201 may switch the display of one recognition image to the display of the other recognition image, in accordance with the application of a display switching instruction.

In addition, in this embodiment, the pixel corresponding to the small blood vessel or the notable blood vessel is displayed by being colored with a color not existing inside the human body, such as a blue-based color or a green-based color, but pixels existing around the pixel may be displayed by being colored with the same color or different colors. By applying such an effect, it is possible to display the small blood vessel portion or the notable blood vessel portion to be intensified (to be thick), and to improve visibility. Note that, only one of the small blood vessel portion and the notable blood vessel portion may be displayed to be intensified, or both portions may be displayed to be intensified.

Further, when the small blood vessel portion or the notable blood vessel portion is colored, a display color (a blue-based color or a green-based color) set for the small blood vessel portion or the notable blood vessel portion and a display color in the operation field image of the background may be averaged, and the blood vessel portions may be displayed by being colored with the averaged color. For example, in a case where the display color set for the blood vessel portion is (0, 0, B1), and the display color of the blood vessel portion in the operation field image of the background is (R2, G2, B2), the control unit 201 may display the blood vessel portions to be colored with a color of (R2/2, G2/2, (B1+B2)/2). Alternatively, weight coefficients W1 and W2 may be introduced, and the recognized blood vessel portion may be displayed by being colored with a color of (W2×R2, W2×G2, W1×B1+W2×B2).

Further, at least one of the small blood vessel portion and the notable blood vessel portion may be displayed to blink. That is, the control unit 201 may repeatedly execute processing of displaying the recognized blood vessel portion only for a first setting time (for example, for 2 seconds) and processing of not displaying the recognized blood vessel portion only for a second setting time (for example, for 2 seconds), alternately, to periodically switch the display and the non-display of the blood vessel portion. The display time and the non-display time of the blood vessel portion may be suitably set. In addition, the display and the non-display of the blood vessel portion may be switched in synchronization with biological information such as the heart rate, or the pulse of the patient.

Further, in this embodiment, the display instruction or the switching instruction is applied by the operation unit 203 of the surgery support device 200, but the display instruction or the switching instruction may be applied by the manipulation unit 11C of the laparoscope 11, or the display instruction or the switching instruction may be applied by a foot switch, a voice input device, or the like, which is not illustrated.

Further, in a case where the notable blood vessel is recognized by the second learning model 320, the surgery support device 200 may enlargedly display a predetermined area including the notable blood vessel. The enlarged display may be performed on the operation field image, or may be performed on another screen.

Further, in this embodiment, the display device 130 displays the small blood vessel and the notable blood vessel to be superimposed on the operation field image, but the detection of the small blood vessel and the notable blood vessel may be notified to the surgeon by a sound or a voice.

Further, in this embodiment, in a case where the notable blood vessel is recognized by the second learning model 320, the control unit 201 may generate a control signal for controlling the energy treatment tool 12 or a medical device such as a surgery robot (not illustrated), and may output the generated control signal to the medical device. For example, the control unit 201 may supply a current to the energy treatment tool 12 to output a control signal for performing the clotting cutting such that the notable blood vessel can be cut while being clotted.

As described above, in this embodiment, the structure of the small blood vessel and the notable blood vessel can be recognized by using the learning models 310 and 320, and the recognized small blood vessel portion and notable blood vessel portion can be displayed to be discriminable by pixel unit, and thus, visual support in the laparoscopic surgery can be performed. Note that, the image generated from the surgery support device 200 may be used not only in the surgery support, but also for education support of a doctor-in-training or the like, or may be used for the evaluation of the laparoscopic surgery. In addition, for example, the image recorded in the recording device 140 during the surgery is compared with the image generated by the surgery support device 200, and whether a tugging manipulation or a peeling manipulation in the laparoscopic surgery is appropriate is determined, and thus, the laparoscopic surgery can be evaluated.

### Embodiment 2

In Embodiment 2, a configuration will be described in which the recognition result of the first learning model 310 is diverted when the training data for the second learning model 320 is generated.

Note that, since the overall configuration of the laparoscopic surgery support system, the internal configuration of the surgery support device 200, and the like are the same as those in Embodiment 1, the description thereof will be omitted.

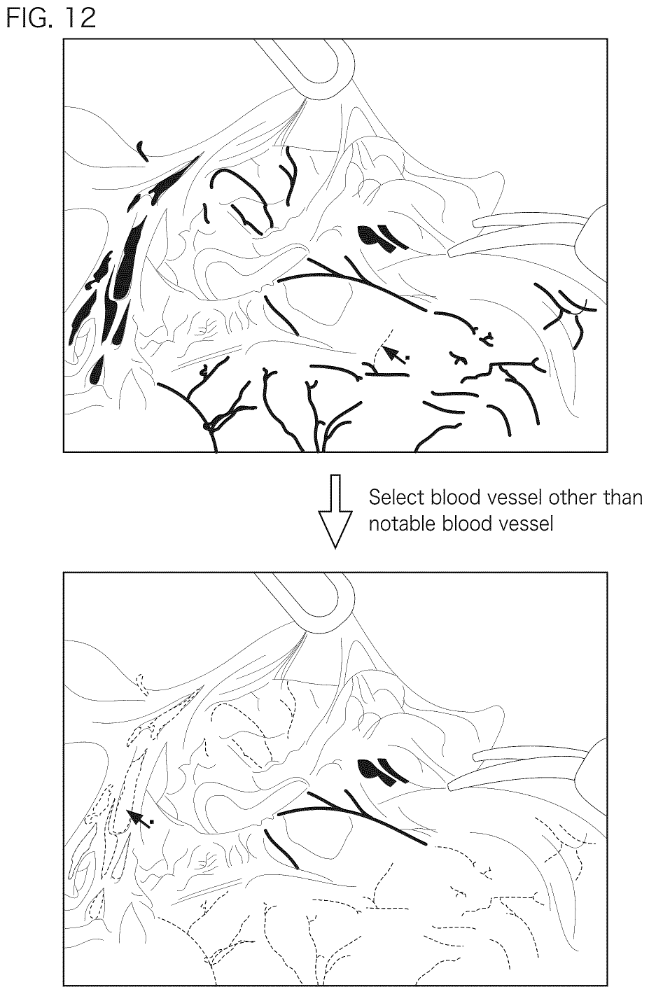

FIG. 12 is an explanatory diagram illustrating a method for generating the training data for the second learning model 320. In Embodiment 1, in the preliminary stage for generating the second learning model 320, the operator performs the annotation by designating the portion corresponding to the notable blood vessel by pixel unit. In contrast, in Embodiment 2, the operator performs the annotation by displaying the recognition result of the small blood vessel by the first learning model 310, selecting a small blood vessel not corresponding to the notable blood vessel among the recognized small blood vessels, and excluding the small blood vessel to leave only the notable blood vessel.

The control unit 201 of the surgery support device 200 recognizes a set of pixels corresponding to the small blood vessel as an area by labeling that the adjacent pixels are the small blood vessel, with reference to the recognition result of the first learning model 310. The control unit 201 receives a selection operation (a click operation or a tap operation of the operation unit 203) with respect to a small blood vessel area not corresponding to the notable blood vessel, among the recognized small blood vessel areas, and thus, excludes the blood vessel other than the notable blood vessel. The control unit 201 designates the pixel of the small blood vessel area that is not selected as the pixel corresponding to the notable blood vessel. A set of the data (the second ground truth data) indicating the position of the pixel corresponding to the notable blood vessel, which is designated as described above, and the original operation field image is stored in the storage unit 202 of the surgery support device 200, as the training data for generating the second learning model 320.

The control unit 201 generates the second learning model 320 by using the training data stored in the storage unit 202. Since a method for generating the second learning model 320 is the same as that in Embodiment 1, the description thereof will be omitted.

As described above, in Embodiment 2, the training data for the second learning model 320 can be generated by diverting the recognition result of the first learning model 310, and thus, a work burden of the operator can be reduced.

Note that, in this embodiment, the notable blood vessel is designated by selecting the small blood vessel to be excluded, but the notable blood vessel may be designated by receiving the selection operation with respect to the small blood vessel corresponding to the notable blood vessel among the small blood vessels recognized by the first learning model 310.

### Embodiment 3

In Embodiment 3, a configuration will be described in which both of the small blood vessel and the notable blood vessel are recognized by using one learning model.

Note that, since the overall configuration of the laparoscopic surgery support system, the internal configuration of the surgery support device 200, and the like are the same as those in Embodiment 1, the description thereof will be omitted.

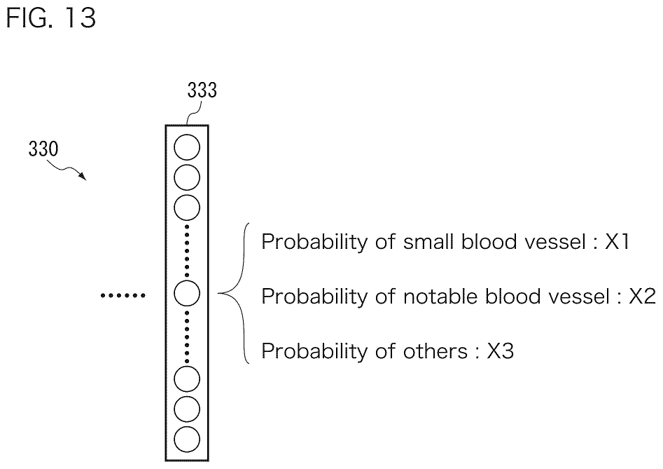

FIG. 13 is an explanatory diagram illustrating the configuration of a softmax layer 333 of the learning model 330 in Embodiment 3. In FIG. 13, for simplicity, only the softmax layer 333 of the learning model 330 is illustrated. The softmax layer 333 outputs a probability to a label set corresponding to each pixel. In Embodiment 3, a label for identifying the small blood vessel, a label for identifying the notable blood vessel, and a label for identifying the others are set. In a case where the probability of the label for identifying the small blood vessel is a threshold value or greater, the control unit 201 of the surgery support device 200 recognizes that the pixel is the small blood vessel, and in a case where the probability of the label for identifying the notable blood vessel is the threshold value or greater, the control unit recognizes that the pixel is the notable blood vessel. In addition, in a case where the probability of the label for identifying the others is the threshold value or greater, the control unit 201 recognizes that the pixel is neither the small blood vessel nor the notable blood vessel.

The learning model 330 for obtaining such a recognition result is generated by training using a data set of the operation field image and ground truth data indicating the position (the pixel) of the small blood vessel portion and the notable blood vessel portion, which are included in the operation field image, in the training data. Since a method for generating the learning model 330 is the same as that in Embodiment 1, the description thereof will be omitted.

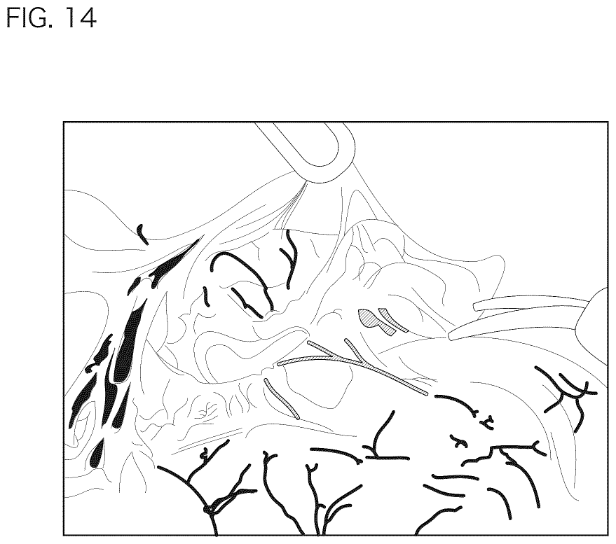

FIG. 14 is a schematic view illustrating a display example in Embodiment 3. The surgery support device 200 in Embodiment 3, the small blood vessel portion and the notable blood vessel portion, which are included in the operation field image, are recognized by u sing the learning model 330, and are displayed on the display device 130 such that the blood vessel portions are determined. In the display example of FIG. 14, for the convenience of drawing, the small blood vessel portion recognized by using the learning model 330 is illustrated with a thick solid line or as an area painted with black, and the notable blood vessel portion is illustrated with hatching. In practice, a portion corresponding to the notable blood vessel may be displayed by being colored with a color not existing inside the human body, such as a blue-based color or a green-based color, by pixel unit, and a portion corresponding to the small blood vessel other than the notable blood vessel may be displayed by being colored with other colors. In addition, notable blood vessel and the small blood vessel other than the notable blood vessel may be displayed with different transmittances. In this case, a relatively low transmittance may be set for the notable blood vessel, and a relatively high transmittance may be set for the small blood vessel other than the notable blood vessel.

As described above, in Embodiment 3, since the small blood vessel portion and the notable blood vessel portion, which are recognized by the learning model 330, are displayed to be discriminable, information useful when performing the tugging manipulation, the peeling manipulation, or the like can be accurately presented to the surgeon.

### Embodiment 4

In Embodiment 4, a configuration will be described in which a display mode is changed in accordance with a confidence of the recognition result with respect to the small blood vessel and the notable blood vessel.

As described in Embodiment 4, the softmax layer 333 of the learning model 330 outputs the probability to the label set corresponding to each pixel. The probability represents the confidence of the recognition result. The control unit 201 of the surgery support device 200 changes the display mode of the small blood vessel portion and the notable blood vessel portion, in accordance with the confidence of the recognition result.

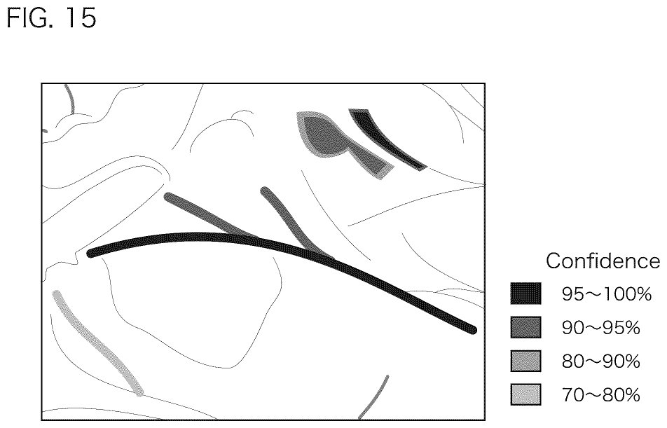

FIG. 15 is a schematic view illustrating a display example in Embodiment 4. FIG. 15 enlargedly illustrates the area including the notable blood vessel. In this example, for the recognition result of the notable blood vessel, the notable blood vessel portion is displayed by changing a concentration in each of a case where the confidence is 70% to 80%, a case where the confidence is 80% to 90%, a case where the confidence is 90% to 95%, and a case where the confidence is 95% to 100%. In this example, the display mode may be changed such that the concentration increases as the confidence increases.

In the example of FIG. 15, the display mode of the notable blood vessel is changed in accordance with the confidence, and similarly, the display mode of the small blood vessel may be changed in accordance with the confidence.

In addition, in the example of FIG. 15, the concentration is changed in accordance with the confidence, but a color or a transmittance may be changed in accordance with the confidence. In a case where the color is changed, the small blood vessel may be displayed with a color not existing inside the human body such as a blue-based color or a green-based color, as the confidence increases, and the small blood vessel may be displayed with a color existing inside the human body, such as a red-based color, as the confidence increases. In addition, in a case where the transmittance is changed, the display mode may be changed such that the transmittance decreases as the confidence increases.

In addition, in the example of FIG. 15, the transmittance is changed in four stages, in accordance with the confidence, but the transmittance may be minutely set, and gradation display may be performed in accordance with the confidence. In addition, the color may be changed, instead of changing the transmittance.

### Embodiment 5

In Embodiment 5, a configuration of displaying an estimated position of the small blood vessel portion that is hidden behind an object such as the surgical tool and is not visually recognizable will be described.

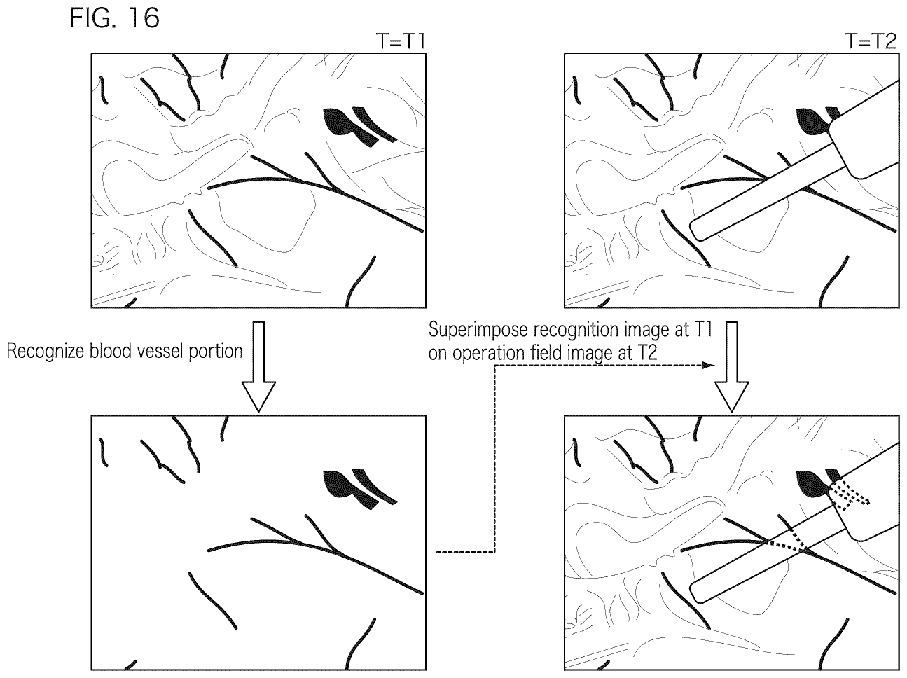

FIG. 16 is an explanatory diagram illustrating a display method in Embodiment 5. As described above, the surgery support device 200 recognizes the small blood vessel portion included in the operation field image by using the learning models 310 and 320 (or the learning model 330). However, in a case where there is the object such as the surgical tool including the energy treatment tool 12 and the forceps 13, and gauze in the operation field of a shooting target, the surgery support device 200 is not capable of recognizing the small blood vessel portion hidden behind the object from the operation field image even in the case of using the learning models 310 and 320 (or the learning model 330). Accordingly, in a case where the recognition image of the small blood vessel portion is displayed to be superimposed on the operation field image, the small blood vessel portion hidden behind the object is not capable of being displayed to be discriminable.

Therefore, the surgery support device 200 according to Embodiment 5, the recognition image of the recognized small blood vessel portion is retained in the storage unit 202 in a state where the small blood vessel portion is not hidden behind the object, and in a case where the small blood vessel portion is hidden behind the object, the recognition image retained in the storage unit 202 is read out and displayed to be superimposed on the operation field image.

In the example of FIG. 16, a time T1 indicates the operation field image in a state where the small blood vessel is not hidden behind the surgical tool, and a time T2 indicates the operation field image in a state where a part of the small blood vessel is hidden behind the surgical tool. Here, the laparoscope 11 is not moved between the time T1 and the time T2, and there is no change in the shot area.

From the operation field image at the time T1, all the small blood vessels appearing in the operation field can be recognized, and the recognition image of the small blood vessel is generated from the recognition result of the learning models 310 and 320 (or the learning model 330). The generated recognition image of the small blood vessel is stored in the storage unit 202.

On the other hand, from the operation field image at the time T2, the small blood vessel not hidden behind the surgical tool, among the small blood vessels appearing in the operation field, can be recognized, but the small blood vessel hidden behind the surgical tool is not recognized. Therefore, the surgery support device 200 reads out the recognition image of the small blood vessel, which is generated from the operation field image at the time T1, from the storage unit 202, and displays the recognition image to be superimposed on the operation field image at the time T2. In the example of FIG. 16, a portion illustrated with a broken line is the small blood vessel portion that is hidden behind the surgical tool and is not visually recognizable, and the surgery support device 200 diverts the recognition image recognized at the time T1, and thus, is capable of displaying the recognition image including the portion to be discriminable.

As described above, in Embodiment 5, since the existence of the small blood vessel that is hidden behind the object such as the surgical tool and is not visually recognizable can be notified to the surgeon, safety during the surgery can be improved.

### Embodiment 6

In Embodiment 6, a configuration will be described in which a running pattern of the blood vessel is predicted, and a blood vessel portion estimated by the predicted running pattern of the blood vessel is displayed to be discriminable.

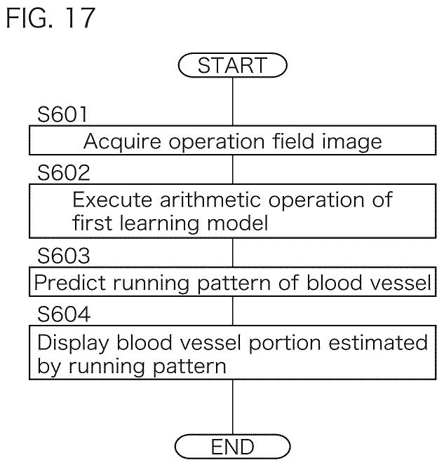

FIG. 17 is a flowchart illustrating the procedure of the processing that is executed by the surgery support device 200 according to Embodiment 6. As with Embodiment 1, the control unit 201 of the surgery support device 200 acquires the operation field image (step S601), inputs the acquired operation field image to the first learning model 310, and executes the arithmetic operation of the first learning model 310 (step S602). The control unit 201 predicts the running pattern of the blood vessel, on the basis of the arithmetic result of the first learning model 310 (step S603). In Embodiment 1, by extracting the pixel in which the probability of the label output from the softmax layer 313 of the first learning model 310 is a first threshold value or greater (for example, 70% or greater), the recognition image of the small blood vessel portion is generated, but in Embodiment 6, by decreasing the threshold value, the running pattern of the blood vessel is predicted. For example, the control unit 201 extracts a pixel in which the probability of the label output from the softmax layer 313 of the first learning model 310 is less than the first threshold value (for example, less than 70%) and is greater than or equal to a second threshold value (for example, 50% or greater), and predicts the running pattern of the blood vessel.

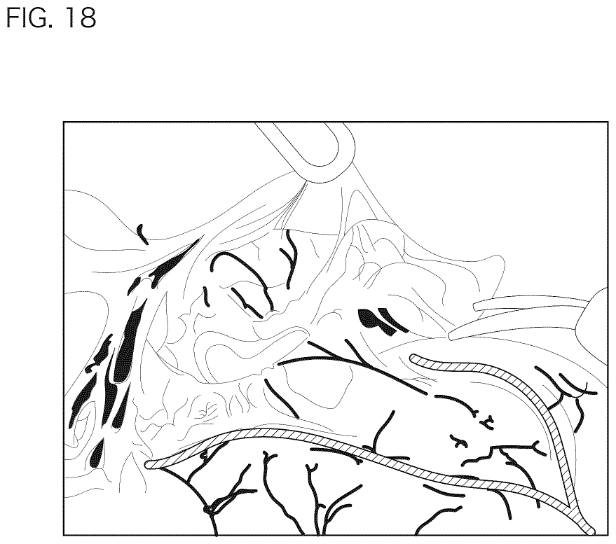

The control unit 201 displays the blood vessel portion estimated by the predicted running pattern to be discriminable (step S604). FIG. 18 is a schematic view illustrating a display example in Embodiment 6. In FIG. 18, the recognized small blood vessel portion is illustrated with a thick solid line (or an area painted with black), and the blood vessel portion estimated by the predicted running pattern is illustrated with hatching. For the convenience of drawing, in the example of FIG. 18, the small blood vessel portion is illustrated with a thick solid line (or as an area painted with black), and the blood vessel portion estimated by the running pattern is illustrated with hatching, but the display may be performed by changing the display mode such as the color, the concentration, and the transmittance.

As described above, in Embodiment 6, since the blood vessel portion estimated by the running pattern of the blood vessel can be displayed together, the visual support in the laparoscopic surgery can be performed.

In this embodiment, by extracting the pixel in which the probability of the label output from the softmax layer 313 is less than the first threshold value (for example, less than 70%) and is greater than or equal to the second threshold value (for example, 50% or greater), the running pattern of the blood vessel is predicted, but a learning model for predicting the running pattern of the blood vessel may be prepared. That is, the learning model trained by using the operation field image obtained by shooting the operation field and ground truth data indicating the running pattern of the blood vessel in the operation field image as the training data may be prepared. The ground truth data may be generated by the expert such as a medical doctor determining the running pattern of the blood vessel while checking the operation field image, and performing the annotation with respect to the operation field image.

### Embodiment 7

In Embodiment 7, a configuration will be described in which a blood flow is recognized on the basis of the operation field image, and a blood vessel is displayed in a display mode according to the amount of blood flow.

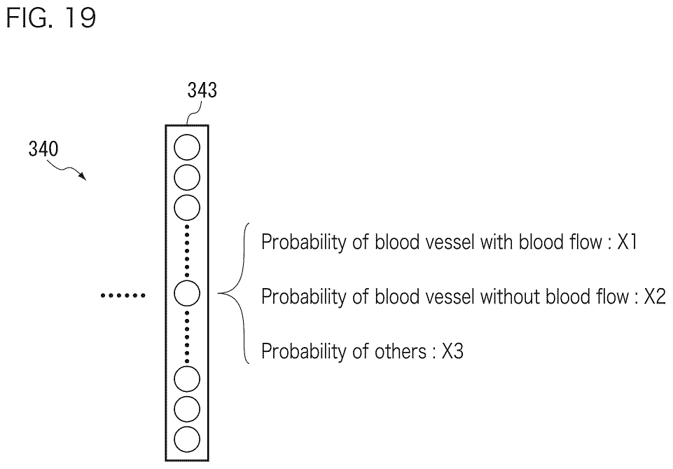

FIG. 19 is an explanatory diagram illustrating the configuration of a softmax layer 343 of a learning model 340 in Embodiment 7. In FIG. 19, for simplicity, only the softmax layer 343 of the learning model 340 is illustrated. The softmax layer 343 outputs the probability to the label set corresponding to each pixel. In Embodiment 7, a label for identifying a blood vessel with a blood flow, a label for identifying a blood vessel without a blood flow, and a label for identifying the others are set. In a case where the probability of the label for identifying the blood vessel with the blood flow is a threshold value or greater, the control unit 201 of the surgery support device 200 recognizes that the pixel is the blood vessel with the blood flow, and in a case where the probability of the label for identifying the blood vessel without the blood flow is the threshold value or greater, the control unit recognizes that the pixel is the blood vessel without the blood flow. In addition, in a case where the probability of the label for identifying the others is the threshold value or greater, the control unit 201 recognizes the pixel is not the blood vessel.

The learning model 340 for obtaining such a recognition result is generated by training using a data set of the operation field image and ground truth data indicating the position (the pixel) of a blood vessel portion with a blood flow and a blood vessel portion without a blood flow, which are included in the operation field image, in the training data. As the operation field image including the blood vessel portion with the blood flow, for example, an indocyanine green (ICG) fluorescence image may be used. That is, a tracer such as ICG having an absorption wavelength in a near-infrared region is injected to an artery or a vein, and fluorescent light emitted when applying near-infrared light is observed to generate a fluorescence image, which may be used as the ground truth data indicating the position of the blood vessel portion with the blood flow. In addition, since the color shade, the shape, the temperature, the blood concentration, the degree of oxygen saturation, and the like of the blood vessel are different between the blood vessel with the blood flow and the blood vessel without the blood flow have, by measuring the color shade, the shape, the temperature, the blood concentration, the degree of oxygen saturation, and the like, the position of the blood vessel portion with the blood flow and the position of the blood vessel portion without the blood flow may be specified, and the ground truth data may be prepared. Since a method for generating the learning model 340 is the same as that in Embodiment 1, the description thereof will be omitted.

Note that, in the learning model 340 illustrated in FIG. 19, the probability that there is a blood flow, the probability that there is no blood flow, and the other probability are output from the softmax layer 343, but the probability may be output in accordance with the amount of blood flow or a blood speed.

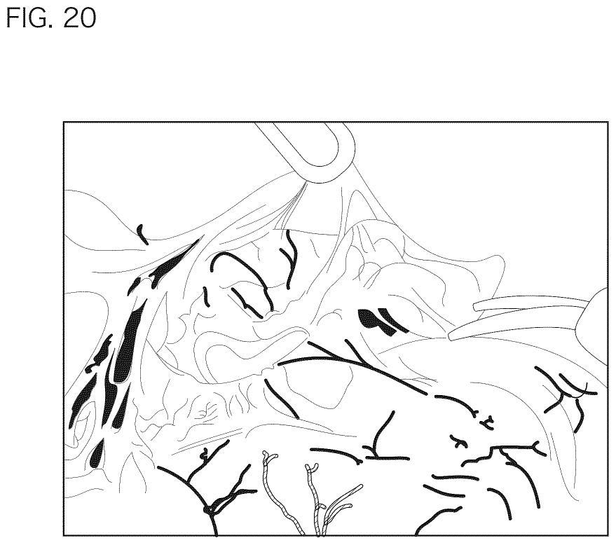

FIG. 20 is a schematic view illustrating a display example in Embodiment 7. The surgery support device 200 in Embodiment 7 recognizes the blood vessel portion with the blood flow and the blood vessel portion without the blood flow by using the learning model 340, and displays the blood vessel portions on the display device 130 to be determinable. In the display example of FIG. 20, for the convenience of drawing, the blood vessel portion with the blood flow is illustrated with a thick solid line or as an area painted with black, and the blood vessel portion without the blood flow is illustrated with hatching, but the blood vessel with the blood flow may be displayed by being colored with a specific color, and the blood vessel without the blood flow may be displayed by being colored with another color. In addition, the blood vessel with the blood flow and the blood vessel without the blood flow may be displayed with different transmittances. Further, either the blood vessel with the blood flow or the blood vessel without the blood flow may be displayed to be discriminable.

As described above, in Embodiment 7, since the blood vessel with the blood flow and the blood vessel without the blood flow are displayed to be discriminable, the visual support in the laparoscopic surgery can be performed.

### Embodiment 8

In Embodiment 8, a configuration will be described in which the blood vessel portion is recognized using a special light image shot by applying special light, and an image of the blood vessel portion recognized using the special light image is displayed as necessary.

The laparoscope 11 in Embodiment 8 has a function of shooting the operation field by applying normal light, and a function of shooting the operation field by applying the special light. Accordingly, the laparoscopic surgery support system according to Embodiment 8 may separately include a light source device (not illustrated) for allowing the special light to exit, or an optical filter for normal light and an optical filter for special light may be switched and applied to light exiting from the light source device 120 to switch and apply the normal light and the special light.

The normal light, for example, is light having a wavelength band (380 nm to 650 nm) of white light. The illumination light described in Embodiment 1 or the like corresponds to the normal light. On the other hand, the special light is illumination light different from the normal light, and corresponds to narrow-band light, infrared light, excitation light, and the like. Note that, in this specification, the discrimination of normal light/special light is merely for convenience and does not emphasize that special light is special compared to the normal light.

In narrow band imaging (NBI), light in two narrowed wavelength bands (for example, 390 to 445 nm/530 to 550 nm) that are easily absorbed in the hemoglobin of the blood is applied to an observation target. Accordingly, the capillary blood vessel of the superficial portion of the mucous membrane, or the like can be displayed to be intensified.

In infra red imaging (IRI), an infrared index agent in which infrared light is easily absorbed is injected intravenously, and then, two infrared light rays (790 to 820 nm/905 to 970 nm) are applied to the observation target. Accordingly, the blood vessel or the like of the deep part of the internal organ, which is difficult to visually recognize in the normal light observation can be displayed to be intensified. As the infrared index agent, for example, ICG can be used.

In auto fluorescence imaging (AFI), excitation light (390 to 470 nm) for observing autofluorescence from a biological tissue and light at a wavelength (540 to 560 nm) that is absorbed in the hemoglobin of the blood are applied to the observation target. Accordingly, two types of tissues (for example, a lesion tissue and a normal tissue) can be displayed to be intensified with different colors.

An observation method using the special light is not limited to the above description, and may be hyper spectral imaging (HSI), laser speckle contrast imaging (LSCI), flexible spectral imaging color enhancement (FICE), and the like.

Hereinafter, the operation field image obtained by shooting the operation field with the application of the normal light will also be referred to as a normal light image, and the operation field image obtained by shooting the operation field with the application of the special light will also be referred to as a special light image.

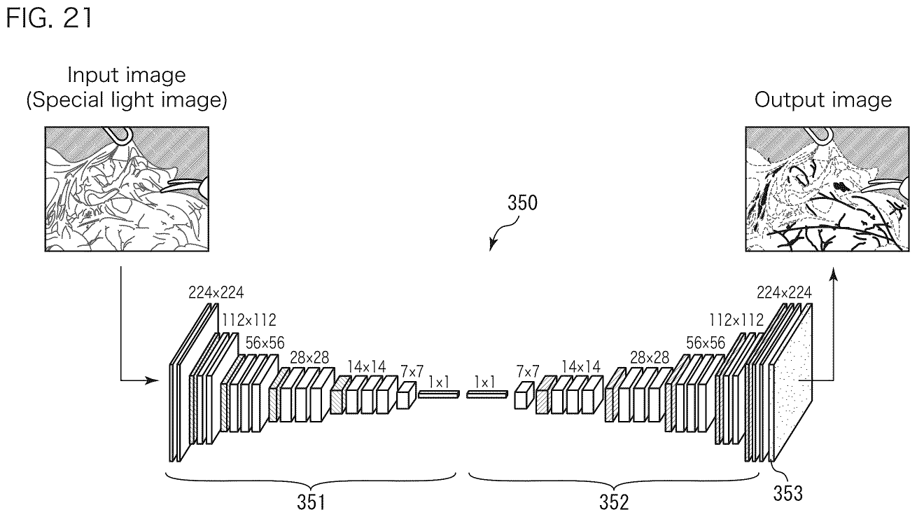

The surgery support device 200 according to Embodiment 8 includes a learning model 350 for a special light image, in addition to the first learning model 310 and the second learning model 320 described in Embodiment 1. FIG. 21 is a schematic view illustrating a configuration example of the learning model 350 for a special light image. The learning model 350 includes an encoder 351, a decoder 352, and a softmax layer 353, and is configured to output an image indicating the recognition result of the blood vessel portion appearing in the special light image with respect to the input of the special light image. Such a learning model 350 is generated by executing training in accordance with a predetermined training algorithm using a data set including an image (the special light image) obtained by shooting the operation field with the application of the special light and data of the position of the blood vessel designated with respect to the special light image by the medical doctor or the like (ground truth data) as training data.

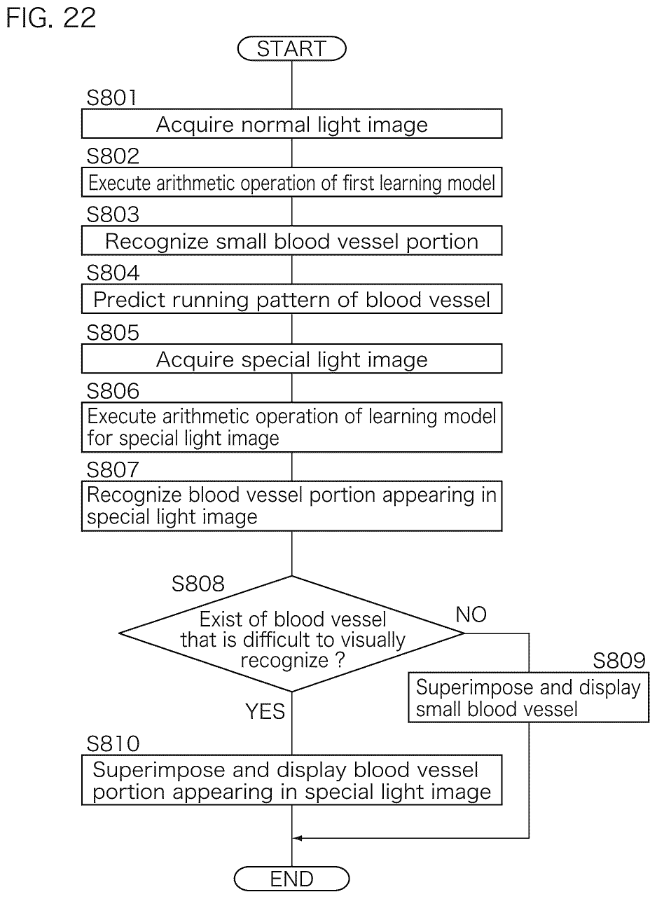

The surgery support device 200 performs the surgery support in an operation phase after the learning model 350 for a special light image is generated. FIG. 22 is a flowchart illustrating the procedure of the processing that is executed by the surgery support device 200 according to Embodiment 8. The control unit 201 of the surgery support device 200 acquires the normal light image (step S801), inputs the acquired normal light image to the first learning model 310, and executes the arithmetic operation of the first learning model 310 (step S802). On the basis of the arithmetic result of the first learning model 310 (step S803), the control unit 201 recognizes the small blood vessel portion included in the normal light image and predicts the running pattern of the blood vessel that is difficult to visually recognize in the normal light image (step S804).

A method for recognizing the small blood vessel is the same as that in Embodiment 1. The control unit 201 recognizes the pixel in which the probability of the label output from the softmax layer 313 of the first learning model 310 is a threshold value or greater (for example, 70% or greater), as the small blood vessel portion. A method for predicting the running pattern is the same as that in Embodiment 6. The control unit 201 predicts the running pattern of the blood vessel that is difficult to visually recognize in the normal light image by extracting the pixel in which the probability of the label output from the softmax layer 313 of the first learning model 310 is less than a first threshold value (for example, less than 70%) and is greater than or equal to a second threshold value (for example, 50% or greater).

The control unit 201 executes the following processing, in parallel with the processing of steps S801 to S804. The control unit 201 acquires the special light image (step S805), inputs the acquired special light image to the learning model 350 for a special light image, and executes an arithmetic operation of the learning model 350 (step S806). On the basis of an arithmetic result of the learning model 350, the control unit 201 recognizes the blood vessel portion appearing in the special light image (step S807). The control unit 201 is capable of recognizing a pixel in which the probability of a label output from the softmax layer 353 of the learning model 350 is a threshold value or greater (for example, 70% or greater), as the blood vessel portion.

Next, the control unit 201 determines whether the existence of the blood vessel that is difficult to visually recognize in the normal light image is detected by the prediction in step S803 (step S808).

In a case where it is determined that the existence of the blood vessel that is difficult to visually recognize is not detected (S807: NO), the control unit 201 outputs the normal light image to the display device 130 from the output unit 205 to be displayed, and displays the recognition image of the small blood vessel portion to be superimposed on the normal light image in a case where the small blood vessel is recognized in step S803 (step S809).

In a case where it is determined that the existence of the blood vessel that is difficult to visually recognize is detected (S807: YES), the control unit 201 outputs the normal light image to the display device 130 from the output unit 205 to be displayed, and displays the recognition image of the blood vessel portion recognized by the special light image to be superimposed on the normal light image (step S810).

As described above, in Embodiment 8, in a case where the existence of the blood vessel that is difficult to visually recognize in the normal light image is detected, the recognition image of the blood vessel portion recognized by the special light image is displayed, and thus, for example, the position of the blood vessel existing in the deep part of the internal organ can be notified to the surgeon, and safety in the laparoscopic surgery can be improved.

Note that, in this embodiment, in a case where the existence of the blood vessel that is difficult to visually recognize in the normal light image is detected, the recognition image of the blood vessel portion recognized by the special light image is automatically displayed, but in a case where the instruction of the surgeon is received through the operation unit 203 or the like, the blood vessel portion recognized by the special light image may be displayed, instead of displaying the small blood vessel portion recognized by the normal light image.

In addition, in this embodiment, the small blood vessel portion is recognized by the normal light image, and the blood vessel portion is recognized by the special light image, but the notable blood vessel portion may be recognized by the normal light image, and the blood vessel portion may be recognized by the special light image, using the second learning model 320.

In this embodiment, a recognition result of the normal light image and a recognition result of the special light image switched and displayed in one display device 130, but the recognition result of the normal light image may be displayed on the display device 130, and the recognition result of the special light image may be displayed on another display device (not illustrated).

In this embodiment, in the control unit 201, the recognition of the small blood vessel portion by the normal light image and the recognition of the blood vessel portion by the special light image are executed, but hardware (such as GPU) different from the control unit 201 may be provided, and in the hardware, the recognition of the blood vessel portion in the special light image may be executed in the background.

### Embodiment 9

In Embodiment 9, a configuration will be described in which the blood vessel portion is recognized by using a combined image of the normal light image and the special light image.

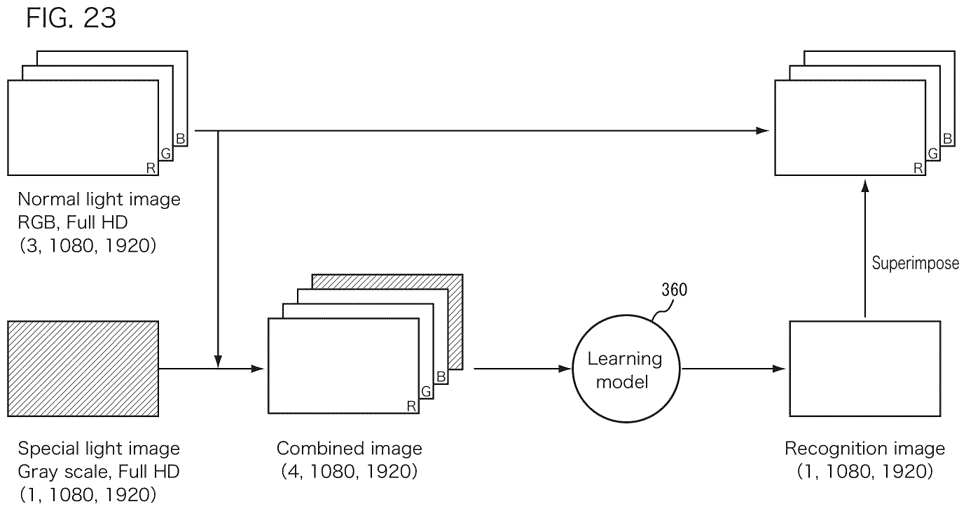

FIG. 23 is an explanatory diagram illustrating the outline of the processing that is executed by the surgery support device 200 according to Embodiment 9. The control unit 201 of the surgery support device 200 acquires the normal light image obtained by shooting the operation field with the application of the normal light and the special light image obtained by shooting the operation field with the application of the special light. In this embodiment, the normal light image, for example, is a full high-definition (HD) RGB image, and the special light image, for example, is a full HD grayscale image.

The control unit 201 generates the combined image by combining the acquired normal light image and special light image. For example, in a case where the normal light image is an image having three-color information (three RGB channels), and the special light image is an image having one-color information (one grayscale channel), the control unit 201 generates the combined image as an image in which four-color information (three RGB channels+one grayscale channel) are compiled into one.

The control unit 201 inputs the generated combined image to a learning model 360 for a combined image, and executes an arithmetic operation of the learning model 360. The learning model 360 includes an encoder, a decoder, and a softmax layer, which are not illustrated, and is configured to output an image indicating the recognition result of the blood vessel portion appearing in the combined image with respect to the input of the combined image. The learning model 360 generates by executing training in accordance with a predetermined training algorithm using a data set including the combined image and data of the position of the blood vessel designated with respect to the combined image by the medical doctor or the like (ground truth data) as training data.

The control unit 201 displays the recognition image of the blood vessel portion obtained by using the learning model 360 to be superimposed on the original operation field image (a normal image).

As described above, in Embodiment 9, the blood vessel portion is recognized by using the combined image, and thus, the existence of the blood vessel that is difficult to visually recognize in the normal light image can be notified to the surgeon, and safety in the laparoscopic surgery can be improved.

Note that, the number of special light images to be combined with the normal light image is not limited to one, and a plurality of special light images with different wavelength bands may be combined with the normal light image.

### Embodiment 10

In Embodiment 10, a configuration will be described in which in a case where the surgical tool approaches or is in contact with the notable blood vessel, such a situation is notified to the surgeon.

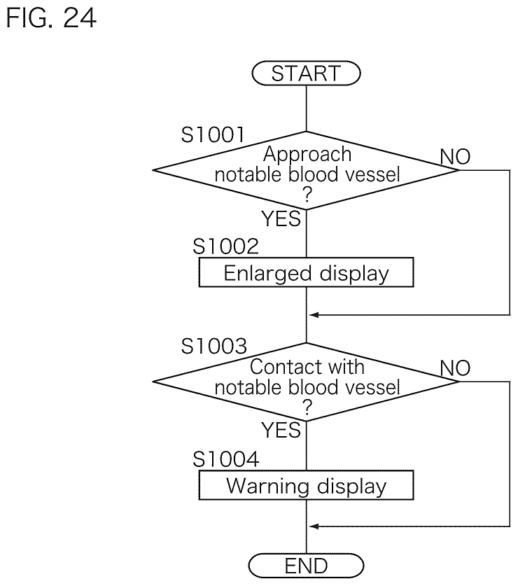

FIG. 24 is a flowchart illustrating an execution procedure of surgery support in Embodiment 10. The control unit 201 of the surgery support device 200 determines whether the surgical tool approaches the notable blood vessel (step S1001). The control unit 201, for example, may calculate an offset distance between the notable blood vessel and the tip of the surgical tool on the operation field image in chronological order, and may determine that the surgical tool approaches the notable blood vessel in a case where it is determined that the offset distance is shorter than a predetermined value. In a case where it is determined that the surgical tool does not approach the notable blood vessel (S1001: NO), the control unit 201 executes the processing subsequent to step S1003 described below.

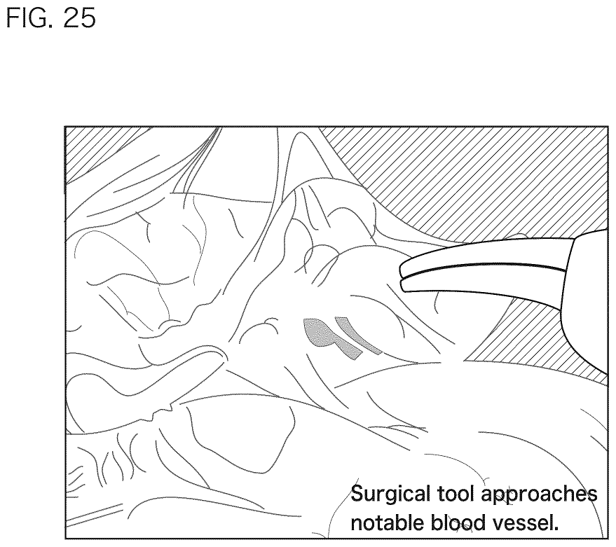

In a case where it is determined that the surgical tool approaches the notable blood vessel (S1001: YES), the control unit 201 enlarged displays the notable blood vessel portion (step S1002). FIG. 25 is a schematic view illustrating an example of the enlarged display. In the example of FIG. 25, an example is illustrated in which the area including the notable blood vessel is enlargedly displayed, and textual information indicating that the surgical tool approaches the notable blood vessel is displayed.

Next, the control unit 201 determines whether the surgical tool is in contact with the notable blood vessel (step S1003). The control unit 201, for example, determines whether the surgical tool is in contact with the notable blood vessel by calculating the offset distance between the notable blood vessel and the tip of the surgical tool on the operation field image in chronological order. In a case where it is determined that the calculated offset distance is zero, the control unit 201 may determine that the surgical tool is in contact with the notable blood vessel. In addition, in a case where there is a contact sensor in the tip portion of the surgical tool, the control unit 201 may determine whether the surgical tool is in contact with the notable blood vessel by acquiring an output signal from the contact sensor. In a case where it is determined that the surgical tool is not in contact with the notable blood vessel (S1003: NO), the control unit 201 ends the processing according to this flowchart.

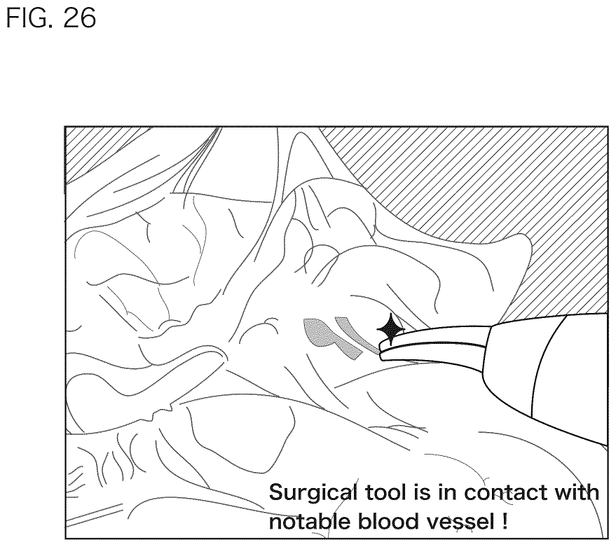

In a case where it is determined that the surgical tool is in contact with the notable blood vessel (S1003: YES), the control unit 201 performs warning display indicating that the surgical tool is in contact with the notable blood vessel (step S1004). FIG. 26 is a schematic view illustrating an example of the warning display. In the example of FIG. 26, an example is illustrated in which textual information indicating that the surgical tool is in contact with the notable blood vessel is displayed by illuminating the surgical tool in contact with the notable blood vessel. A sound or vibration warning may be performed, together with the warning display or instead of the warning display.

Note that, in this embodiment, in a case where the surgical tool is in contact with the notable blood vessel, the warning display is performed, but in a case where the presence or absence of bleeding due to the damage of the notable blood vessel is determined, and it is determined that there is the bleeding, the warning may be performed. For example, in a case where the number of red pixels within the predetermined area including the notable blood vessel is counted in chronological order, and the number of red pixels increases by a certain amount or more, the control unit 201 is capable of determining that there is the bleeding.

The embodiments disclosed herein are considered illustrative in all respects but not restrictive. The scope of the present invention is indicated by the claims but not the meaning described above, and is intended to include all modifications within the meaning and the scope equivalent to the claims.

It is noted that, as used herein and in the appended claims, the singular forms “a”, “an”, and “the” include plural referents unless the context clearly dictates otherwise.

1-25. (canceled)

26. A non-transitory computer readable recording medium storing a computer program for causing a computer to execute processing of:
acquiring an operation field image obtained by shooting an operation field of a scopic surgery; and
distinctively recognizing blood vessels included in the acquired operation field image and a notable blood vessel among the blood vessels by using a learning model trained to output information relevant to a blood vessel when the operation field image is input.

27. The non-transitory computer readable recording medium according to claim 26, storing the computer program for causing the computer to execute processing of:
displaying a blood vessel portion recognized from the operation field image and a notable blood vessel portion on the operation field image to be discriminable.

28. The non-transitory computer readable recording medium according to claim 27, storing the computer program for causing the computer to execute processing of:
displaying both of the blood vessel portions to be switchable.

29. The non-transitory computer readable recording medium according to claim 27 storing the computer program for causing the computer to execute processing of:
displaying both of the blood vessel portions in different display modes.

30. The non-transitory computer readable recording medium according to claim 27, storing the computer program for causing the computer to execute processing of:
periodically switching display and non-display of at least one recognized blood vessel portion.

31. The non-transitory computer readable recording medium according to claim 27, storing the computer program for causing the computer to execute processing of:
applying a predetermined effect to the display of the at least one recognized blood vessel portion.

32. The non-transitory computer readable recording medium according to claim 27, storing the computer program for causing the computer to execute processing of:
calculating a confidence of a recognition result of the learning model; and
displaying at least one blood vessel portion in a display mode according to the calculated confidence.

33. The non-transitory computer readable recording medium according to claim 26, storing the computer program for causing the computer to execute processing of:
displaying an estimated position of a blood vessel portion hidden behind other objects, with reference to a recognition result of the learning model.

34. The non-transitory computer readable recording medium according to claim 26, storing the computer program for causing the computer to execute processing of:
estimating a running pattern of the blood vessel by using the learning model; and
displaying an estimated position of a blood vessel portion not appearing in the operation field image, on the basis of the estimated running pattern of the blood vessel.

35. The non-transitory computer readable recording medium according to claim 26, wherein
the learning model is trained to output information relevant to the blood vessel not existing in a central visual field of a surgeon, as a recognition result of the notable blood vessel.

36. The non-transitory computer readable recording medium according to claim 26, wherein
the learning model is trained to output information relevant to a blood vessel existing in the central visual field of the surgeon, as the recognition result of the notable blood vessel.

37. The non-transitory computer readable recording medium according to claim 26, wherein
the learning model is trained to output information relevant to a blood vessel in a state of tension, and
the computer program causes the computer to further execute processing of recognizing a blood vessel portion in a state of tension as the notable blood vessel, on the basis of the information output from the learning model.

38. The non-transitory computer readable recording medium according to claim 26, storing the computer program for causing the computer to execute processing of:
recognizing a blood flow flowing the blood vessel included in the operation field image by using a learning model for recognizing a blood flow trained to output information relevant to a blood flow, in accordance with the input of the operation field image; and
displaying a blood vessel recognized by using a learning model for recognizing a blood vessel in a display mode according to an amount of blood flow, with reference to a recognition result of the blood flow of the learning model.

39. The non-transitory computer readable recording medium according to claim 26, storing the computer program for causing the computer to execute processing of:
acquiring a special light image obtained by shooting the operation field by emitting another illumination light different from illumination light for the operation field image;
recognizing a blood vessel portion appearing in the special light image by using a learning model for a special light image trained to output information relevant to a blood vessel appearing in the special light image when the special light image is input; and
displaying the recognized blood vessel portion to be superimposed on the operation field image.

40. The non-transitory computer readable recording medium according to claim 29, storing the computer program for causing the computer to execute processing of:
displaying the blood vessel portion recognized from the operation field image and the blood vessel portion recognized from the special light image to be switchable.

41. The non-transitory computer readable recording medium according to claim 26, storing the computer program for causing the computer to execute processing of:
acquiring a special light image obtained by shooting the operation field by emitting another illumination light different from illumination light for the operation field image;
generating a combined image of the operation field image and the special light image;
recognizing a blood vessel portion appearing in the combined image by using a learning model for a combined image trained to output information relevant to a blood vessel appearing in the combined image when the combined image is input; and
displaying the recognized blood vessel portion to be superimposed on the operation field image.

42. The non-transitory computer readable recording medium according to claim 26, storing the computer program for causing the computer to execute processing of:
detecting bleeding, on the basis of the operation field image; and
outputting warning information when the bleeding is detected.

43. The non-transitory computer readable recording medium according to claim 26, storing the computer program for causing the computer to execute processing of:
detecting approach of a surgical tool, on the basis of the operation field image; and
displaying the notable blood vessel to be discriminable when the approach of the surgical tool is detected.

44. The non-transitory computer readable recording medium according to claim 26, storing the computer program for causing the computer to execute processing of:
enlarged displaying a blood vessel portion recognized as the notable blood vessel.

45. The non-transitory computer readable recording medium according to claim 26, storing the computer program for causing the computer to execute processing of:
outputting control information to a medical device, on the basis of the recognized blood vessel.

46. A learning model generating method that causes a computer to execute processing of:
acquiring training data including an operation field image obtained by shooting an operation field of a scopic surgery, first ground truth data indicating blood vessel portions included in the operation field image, and second ground truth data indicating a notable blood vessel among the blood vessel portions; and
generating a learning model for outputting information relevant to a blood vessel, on the basis of a set of the acquired training data, when the operation field image is input.

47. The learning model generating method according to claim 46 that causes the computer to execute processing of:
generating a first learning model and a second learning model individually, wherein
the first learning model outputs information relevant to blood vessels included in the operation field image when the operation field image is input, and
the second learning model outputs information relevant to a notable blood vessel among the blood vessels included in the operation field image when the operation field image is input.

48. A learning model generating method that causes a computer to execute processing of:
acquiring training data including an operation field image obtained by shooting an operation field of a scopic surgery, and first ground truth data indicating blood vessel portions included in the operation field image;
generating a first learning model for outputting information relevant to a blood vessel, on the basis of a set of the acquired training data, when the operation field image is input;
generating second ground truth data by receiving designation for a notable blood vessel among the blood vessel portions in the operation field image recognized by using the first learning model; and
generating a second learning model for outputting information relevant to the notable blood vessel, on the basis of a set of training data including the operation field image and the second ground truth data, when the operation field image is input.

49. The learning model generating method according to claim 46, wherein
the notable blood vessel among the blood vessel portions included in the operation field image is a blood vessel portion in a state of tension.

50. A surgery support device, comprising:
a processor; and
a storage storing instructions causing the processor to execute processes of:
acquiring an operation field image obtained by shooting an operation field of a scopic surgery;
recognizing blood vessels included in the acquired operation field image and a notable blood vessel among the blood vessels by using a learning model trained to output information relevant to a blood vessel when the operation field image is input; and
outputting support information relevant to the scopic surgery, on the basis of a recognition result.
